(19) United States
(12) Patent Application Publication
(10) Pub. No.: US 2009/0035815 A1
(43) Pub. Date: Feb. 5, 2009

(54) Synthetic Gene for Enhanced Expression in E. Coli

(76) Inventors: Laxmi Srinivas Rao, Mumbai (IN); Monsur Borbhuiya Ahmed, Mumbai (IN); Milind Niphadkar, Mumbai (IN); Dinesh Paliwal, Mumbai (IN); Rakesh Shekhawat, Mumbai (IN); Aruna Khare, Mumbai (IN); Radhika Samant, Mumbai (IN); Priti Thakur, Mumbai (IN); Anjali Chutke, Mumbai (IN); Neelesh Surlikar, Mumbai (IN); Sagar Zawar, Mumbai (IN); Shrikant Mishra, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 12/126,319

(22) Filed: May 23, 2008

(30) Foreign Application Priority Data
Jun. 29, 2007 (IN) 1247/MUM/2007

(51) Int. Cl. C12P 21/06; C12N 15/62; C12P 41/00; C07H 21/04; C12N 15/12

(52) U.S. Cl. 435/68.1; 435/69.1; 536/23.4; 435/280; 536/24.33; 536/23.5

(57) ABSTRACT

A novel nesiritide synthetic cDNA chimera encoding human b-type natriuretic peptide (hBNP) or nesiritide and a process for the preparation of the said novel chimera. Further, the inventors disclose the use of nesiritide synthetic cDNA chimera to obtain an expressible construct to produce mature nesiritide. Particularly, the inventors disclose an application of recombinant cloning method to prepare an ORF of a nesiritide chimeric construct, which is simultaneously codon optimized for E. coli and has optimal RNA stability. The inventors also provide a process for large scale purification of nesiritide by pH precipitation and chromatography.

### RELATED APPLICATIONS

This application claims priority from Indian Application 1247/MUM/2007, filed on 29 Jun. 2007.

### TECHNICAL FIELD

Our invention also relates to a novel nesiritide synthetic cDNA chimera as shown in Seq. ID No. 3 encoding human b-type natriuretic peptide (often called “hBNP” or “nesiritide”) and a process for the preparation of said novel chimera. Further the invention relates to the use of nesiritide synthetic cDNA chimera to obtain an expressible construct to produce mature nesiritide. Particularly, the invention relates to an application of recombinant cloning method to prepare an ORF of a nesiritide chimeric construct, which is simultaneously codon optimized for E. coli and has optimal RNA stability.

Our invention further relates to a process for purification of peptide, in particular nesiritide by pH precipitation and chromatography. Our method can be useful to manufacture a variety of disulfide-bond containing polypeptides produced by recombinant DNA technology.

### BACKGROUND

The peptide, b-type natriuretic peptide or BNP, occurs in human as a 32-amino acid peptide which is produced by the cleavage of a 134-amino acid precursor protein. The actual site of synthesis for brain type human natriuretic peptide is the ventricular myocardium (Mukoyama et al., 1991). BNP is constitutively released from ventricular myocyte as a preprohormone of 134 amino acids, which is cleaved into a proBNP hormone. When secreted, it is further cleaved to 76 amino acids N-terminal BNP and a 32 amino acids active hormone and released into the blood (Hama et. al., 1995). B-type natriuretic peptide, also known as nesiritide, is shown to improve heart function without direct cardiac stimulation and to decrease levels of neurohormones associated with increased mortality and acceleration of the progression of heart failure. Accordingly, it is approved by US FDA for treatment of patients with acute decompensated congestive heart failure who have dyspnea at rest or with minimal activity. In view of the clinical advantages fulfiled by nesiritide, a need for development of efficient methods of producing nesiritide exists.

Different technologies are available for the production of peptides and proteins such as extraction from natural sources (Hipkins and Brownson, 2000), production by rDNA technology (Gill et al., 1996), production in cell free expression systems (Katzen et al., 2005), production in transgenic animals (Wright et al., 1991), production in plants (Cunningham and Porter, 1997), production by chemical synthesis (Du Vigneaud et a1.1953; Merrifield, 1963) and by enzyme technology using proteolytic enzymes under conditions of displacement of the equilibrium of the reaction towards the formation of peptide bond (Feliu et al. 1995). The size of the molecule determines the technology most suitable for its production.

Nesiritide can be synthesized chemically by means well known in the art such as solid-phase peptide synthesis (SPPS). U.S. Pat. No. 5,114,923 teaches solid phase peptide synthesis of brain natriuretic peptide and natriuretic related peptides wherein the synthesis commences from the carboxy terminal end of the peptide using t-Butyloxycarbonyl (Boc) as protecting group for α-amino groups and use of chloromethylated polystyrene resin as the solid support. US2006/0148699 patent application exemplifies synthesis of nesiritide carried out by a regular stepwise Fmoc solid phase peptide synthesis procedure starting from 2-chloro-trityl resin. Fmoc protected amino acids are activated in situ using TBTU (N,N,N′,N′-Tetramethyl-O-(Benzotriazol-1-yl) uronium tetrafluoroborate) and HOBt (N-hydroxybenzotriazole) and are deprotected by 20% piperidine in dimethylformamide solution. The cleavage cocktail used is 95% TFA (Trifluoroacetic acid), 2.5% TIS (Triisopropylsilane), 2.5% EDT (Ethane dithreitol) and the peptide is either precipitated or filtered and dried in vacuum. Even though chemical synthesis is the most mature technology for peptide synthesis, lack of specificity and environmental burden are severe drawbacks whereas recombinant expression of peptides overcomes the said drawbacks.

Recombinant expression is the preferred mode for synthesis of proteins or peptides. The choice of an expression system for the high-level production of recombinant proteins depends on many factors such as cell growth characteristics, expression levels, intracellular and extracellular expression, posttranslational modifications, and biological activity of the protein of interest, as well as regulatory issues in the production of therapeutic proteins (Goeddel, D. V. 1990. Systems for heterologous gene expression. Methods Enzymol. 185: 3-7; Hodgson, J. 1993. Expression systems: a user's guide. Bio/Technology 11: 887-893). In addition, the selection of a particular expression system requires a cost breakdown in terms of process, design, and other economic considerations. The relative merits and demerits of bacterial, yeast, insect, and mammalian expression systems are well known in the art. However, bacterial systems remain most attractive due to low cost, high productivity, and rapid use. Particularly, overexpression of target genes as heterologous proteins in Escherichia coli as a host is often the method of choice, because of extensive knowledge of E. coli genetics, availability of versatile vector systems and host strains, the ease of use, low costs and high expression levels, often exceeding more than 30% of total cellular protein (G. Hannig, S. C. Makrides, Trends Biotechnol. 16 (1998) 54-60; S. C. Makrides, Microbiol. Rev. 60 (1996) 512-538; F. Baneyx, Curr. Opin. Biotechnol. 10 (1999), 411-421).

Recombinant production of Nesiritide in E. coli is covered by basic innovator patents, U.S. Pat. No. 5,114,923, U.S. Pat. No. 5,948,761 and U.S. Pat. No. 5,674,710 which disclose cDNA sequence encoding porcine brain natriuretic peptide (pBNP) and related genes encoding canine and human peptides with natriuretic activity. The invention relates to use of standard techniques to locate the intron in porcine cDNA which is incompletely processed and manipulation of the said sequence to obtain an ORF for pBNP. The invention encompasses construction of genomic and cDNA libraries from readily accessible atrial tissues as source for pBNP, use of a porcine BNP probe to identify canine brain natriuretic peptide probe, subcloning the canine gene into pBR322 vector and use of the resultant pdBNP probe to screen blots of digested human genomic DNA to obtain the human BNP gene, cloning in an expression vector and expression of the said gene of interest in E. coli. One major limitation in the use of cDNA methods is that the cDNA methods do not always prove satisfactory owing to the termination of transcription short of the entire sequence and/or the desired sequence due to an intron and are also accompanied by naturally occurring precursor leader or signal DNA. Thus, these attempts often result in incomplete protein product and/or protein product in non-cleavable conjugate form (Villa-Komaroff et al., Proc. Natl. Acad. Sci. (USA) 75, 3727 (1978) and Seeburg et al., Nature 276, 795 (1978). U.S. Pat. No. 5,114,923, U.S. Pat. No. 5,948,761 and U.S. Pat. No. 5,674,710 also report a similar problem which additionally requires an effort to locate and splice the intron to have an ORF for hBNP. Another problem encountered is the difficulty to collect total RNA from human tissue sources such as the atrium and brain, wherein the major constraints are availability in small quantity and isolation of mRNA from the total RNA pool.

Also high level expression of peptides in E. coli is not routinely achieved. J. F. Kane, Curr. Opin. Biotechnol. 6 (1995) 494-500, teaches many reported causes preventing efficient heterologous protein production in E. coli characterized by biased codon usage, gene product toxicity, solubility, mRNA secondary structure, and mRNA stability. In addition, other cause of concern is rare codon gene expression which can lead to translational errors as a result of ribosomal stalling at a position requiring incorporation of amino acids coupled to minor tRNAs, or even at sites requiring major tRNAs, but which are depleted because of overutilization of a particular amino acid (D. E. McNulty, B. A. Claffee, M. J. Huddleston, J. F. Kane, Protein Expr. Purif. 27, (2003) 365-374; T. L. Calderone, R. D. Stevens, T. G. Oas, J. Mol. Biol. 262 (1996) 407-412). The mistranslational events related to rare tRNAs are observed as codon misreadings and as processing errors and they manifest themselves as amino acid substitutions or frameshift events. Specifically, the rare arginine (AGG, AGA, CGG, and CGA), leucine (CUA), isoleucine (AUA), and proline codons (CCC) often lead to frameshift errors and ultimately to undesired products (D. E. McNulty, B. A. Claffee, M. J. Huddleston, J. F. Kane, Protein Expr. Purif. 27, (2003) 365-374; T. L. Calderone, R. D. Stevens, T. G. Oas, J. Mol. Biol. 262 (1996) 407-412; B. Q. Wang, L. Lei, Z. F. Burton, Protein Expr. Purif. 5 (1994) 476-485; S. L. Martin, B. Vrhovski, A. S. Weiss, Gene 154 (1995) 159-166; E. Goldman, A. H. Rosenberg, G. Zubay, F. W. Studier, J. Mol. Biol. 245 (1995) 467-473; R. A. Spanjaard, J. van Duin, Proc. Natl. Acad. Sci. USA 85 (1988) 7967-7971; J. Sipley, E. Goldman, Proc. Natl. Acad. Sci. USA 90 (1993) 2315-2319). Literature cites the commercial availability of many E. coli hosts wherein the low frequency tRNAs have been suitably optimized. The most preferred systems to alleviate the problems associated with rare codon gene expression are either the tight transcriptional regulation or heterologous expression of tRNAs.

Another approach well known in the art for high level expression of heterologous protein is expression of the protein of interest as fusion protein in either soluble or inclusion body form in a prokaryotic host preferably E. coli. Literature teaches that the production of peptides having less than 50 amino acids in length by expression of the peptide-encoding DNA in recombinant host cell such as E. coli is commonly plagued by the problem of enzymatic degradation of the expressed peptide within the host cell, resulting in partial or complete loss of the peptide. The fusion protein forms inclusion bodies within the cell, within which the peptide is protected from degradation by proteolytic enzymes. Large fusion proteins exhibit a disadvantage in prokaryotic expression systems as there is a concurrent increase in the probability of incomplete transcription or translation by premature termination or internal initialization. U.S. Pat. No. 6,303,340 teaches hBNP (1-32) and hBNP (2-32) expression as a fusion protein with a modified N-terminal chloramphenicol acetyl transferase (CAT) sequence, the junction of the fusion partner and the b-type natriuretic peptide forms an Asp-Ser dipeptide susceptible to acid cleavage wherein CAT-BNP fusion protein is produced in E. coli in a vector under the control of PhoA promoter and contains tetracycline resistant gene. JP5207891 discloses a process for preparation of nesiritide in the form of fusion proteins wherein glutamic acid is used as a linker and the fusion protein is subsequently cut enzymatically. CN1594581 teaches the artificial synthesis of human nesiritide BNP wherein the fusion protein is expressed using mutated CMP-3-deoxy-D-manna-octulosonic acid synthase gene as the fusion partner with enterokinase cut site at the junction, cloning and expression into Pichia under the control of AOX promoter induced by methanol. Besides literature reporting several advantages offered, the inherent disadvantages of slow growth and poor transformation efficiency of Pichia as compared to E. coli and moreover proteolytic degradation of the expressed proteins remains a challenge restricting the commercial exploitation of the Pichia expression platform.

There are numerous reports wherein β-galactosidase is the preferred fusion partner for better expression of heterologous proteins in E. coli. U.S. Pat. No. 5,670,340 teaches a process to express a gene coding for a fusion protein with isoelectric point between 4.9 and 6.9 wherein the fusion partner or protective peptide comprises a 90-210 amino acid fragment, preferably 97 amino acids E. coli β-galactosidase fragment spaced by a linker from the N terminal end of target protein selected from calcitonins or C-type natriuretic peptides and the improvement consisting of amino acids mutation in E. coli β-galactosidase to change a cysteine to a serine residue and four glutamic residues to aspartic acid residues for target peptide expression in large amount. U.S. Pat. No. 5,670,340 discloses that in the case of producing a target peptide as a portion of a fusion protein, there is no established theory as to how large the protective peptide should be despite the size of the protective peptide being closely related to the stability of the fusion protein inside the microorganism. In addition it emphasizes that there are no established methods for large-scale production on an industrial scale by expressing fusion protein as stable inclusion bodies in microorganism. Hence, still it is of commercial interest to construct several families of plasmids that code for truncated β-galactosidase fragments as chimeric agents, and to determine the minimum length of a truncated β-galactosidase that can produce a stable fusion protein.

Numerous literature reports the formation of inclusion bodies to be advantageous for large scale production of fusion proteins wherein the target peptide is shielded from proteolytic degradation. U.S. Pat. No. 6,303,340 relates to acid cleavage of the fusion protein formed in inclusion bodies in the absence of chaotropes. The patent justifies the use of acid cleavage as against the high cost of enzymatic cleavage. It also teaches the advantage of the relative high pI of the b-type natriuretic peptide for the purification of the acid cleaved peptide by ion exchange chromatography. Pollitt et al (U.S. Pat. No. 6,303,340) discloses the expression of nesiritide as a fusion with a modified chloramphenicol acetyl transferase sequence, the modification comprising of replacement of one or more acidic or basic amino acid residues with hydrophobic residues so as to achieve charge balance and to shift the pI of the fusion protein preferably between 6.5 and 7.5. However, chemical methods generally result in cleavage directed towards an individual amino acid (Grant, G. (Ed.) (1992) in “Synthetic Peptide, A User's Guide,” pp. 234-235, W. H. Freeman, NY.) and are not well suited to polypeptides where the target amino acid appears repeatedly. This limitation generally excludes chemical cleavage from consideration for affinity tag removal from larger polypeptides and proteins. It is well known in the art that the preferred method for affinity tag removal from complex target protein is proteolytic cleavage. Acid cleavage has the limitation of recognizing a dipeptide and cleaving at internal sites having the recognition sequence within the target protein resulting in loss of biological activity of the target protein as compared to enterokinase, a proteolytic enzyme, which has the advantage of recognizing a five amino acid sequence thus reducing the likelihood of a secondary cleavage event within the target protein. The present invention encompasses the problem of internal cleavage by use of enterokinase, which cleaves the target protein on the C-terminal side of the recognition sequence (DDDDK) allowing complete removal of affinity tag sequences. The present invention thus avoids harsh conditions of chemical cleavage of the target protein.

In another report, Ziyong Sun et al. (Prot. Exp. Purif. 2005 September; 43(1): 26-32) mention the use of an intein mediated cleavage process to release B-type natriuretic peptide from the fusion protein. The fusion protein, expressed as inclusion bodies is isolated and solubilized in 6M guanidine hydrochloride and thereafter refolded using redox buffer containing reduced and oxidized glutathione. The dilute solution containing refolded fusion protein is bound to chitin beads and the release of peptide is affected by shift in pH from 9.0 to 7.0 and incubating the solution at 25° C. for 16 hours.

Another persistent challenge in recombinant expression of proteins is the rapid and economical purification of proteins. Protein purification typically involves several chromatographic steps, each of which must be individually optimized for each product protein. Each step can be costly and time consuming, and inevitably decreases the final yield of the product (Freitag and Horvath 1996). In the large-scale manufacture of recombinant proteins for industrial and therapeutic use, downstream purification is very costly and can account for up to 80% of the total production cost (Hearn and Acosta 2001). Development of simple and reliable methods for protein purification, which can be applied to any products at laboratory to manufacturing scale is therefore an important goal in bioseparation technology development. The present invention is directed to an easily scaleable process for optimized production of nesiritide by taking advantage of the high pI of the target protein, employing pH precipitation and hydrophobic interaction chromatography as final polishing step for recovery of the target protein.

Hydrophobic interaction chromatography (HIC) involves the use of high molarities of salt in the protein solution but at concentrations that are below their precipitation points. At these salt concentrations, certain ligands, which under normal salt conditions would not adsorb these proteins, become excellent adsorbents. The principle for protein adsorption to HIC is complementary to ion exchange and gel filtration chromatography methods. The advantage of the present invention is use of an aqueous phase elution by HIC without the use of organic solvents for nesiritide purification.

Commercial exploitation of nesiritide in view of the clinical advantages offered requires the development of an improved process that targets optimized and enhanced protein expression by gene manipulations, easily scaleable, cost-effective and less energy intensive down stream process for efficient recovery of the nesiritide. The present invention has been accomplished in order to deal with aforementioned drawbacks associated with prior art processes for the expression of recombinant nesiritide. This objective has been now successfully achieved by the inventors developing an improved process for synthesis of recombinant nesiritide described in entirety in the present application.

### SUMMARY

The prior art relating to recombinant DNA technology teaches various DNA leader sequences (e.g., the E. coli β-gal gene) which increase polypeptide expression efficiency in recombinant systems. We have found that one can modify a DNA leader so that it both increases expression, and also helps to isolate the resulting product.

The present invention discloses a novel hBNP synthetic cDNA chimera as shown in Seq. ID No. 3 encoding human b-type natriuretic peptide (hBNP) or nesiritide and a process for the preparation of the said novel chimera. Further the invention discloses the use of nesiritide synthetic cDNA chimera to obtain an expressible construct to produce mature nesiritide. Particularly, the invention discloses an application of recombinant cloning method to prepare an ORF of a nesiritide chimeric construct, which is simultaneously codon optimized for E. coli and has optimal RNA stability. The invention further discloses a process for large scale purification of peptide, in particular nesiritide, by pH precipitation and chromatography.

One aspect of the present invention provides an improved process for synthesis of Nesiritide as set forth in Seq. ID NO:2 comprising:
i. preparing a synthetic cDNA construct as set forth in Seq. ID No. 1 encoding ORF of Nesiritide polynucleotide by iterative optimization of RNA transcript free energy operably in the range of (−)30 Kilo calories to (−)300 Kilo calories per mole;
ii. fusing said polynucleotide of step i) with a fusion partner consisting essentially of 41 amino acids from the N-terminal region of E. coli β-galactosidase and an affinity handle operably linked to regulatory elements cloned in an expression vector;
iii. expressing the synthetic cDNA chimera as set forth in Seq. ID No. 3 in a host by culturing the host cells in the growth medium under appropriate conditions to yield Nesiritide chimeric protein; and
iv isolating and purifying the Nesiritide obtained from step iii.

Another aspect of the invention provides a synthetic cDNA construct as set forth in Seq. ID NO: 1 prepared by sequential PCR cloning technique, preferably by primer driven codon optimization of the gene.

Still another aspect of the invention provides gene-specific primers selected from Seq. ID NO:4 to Seq. ID NO:21.

Another aspect of the invention provides an expression vector consisting of synthetic cDNA chimeric sequence as set forth in Seq. ID NO. 3 wherein the expression vector is pET-RAZ-6-N having sequence as set forth in Seq. ID NO:22 with cDNA chimeric sequence from 5315 bp to 5607 bp.

Still another aspect of the invention provides the affinity handle to be selected from maltose-binding protein, poly Histidine and staphylococcal protein wherein said affinity handle is polyhistidine.

Yet another aspect of the invention discloses the host as E. coli wherein E. coli harbors the expression vector pET-RAZ-6-N as set forth in Seq. ID NO:22 having cDNA chimeric sequence from 5315 bp to 5607 bp, said vector comprising synthetic cDNA chimeric sequence as set forth in Seq. ID NO:3.

Further aspect of the invention discloses the expression of synthetic cDNA chimera as set forth in Seq. ID No. 3 encoding Nesiritide which is under the control of a promoter selected from a group consisting of ara_{BAD}, trp, T7, lac, pho, and trc, wherein the promoter is T7.

Still further aspect of the invention discloses growth medium for culturing host cells comprising an inducer selected from a group consisting of Isopropyl-beta-D-thiogalactopyranoside (IPTG), lactose, maltose, arabinose and arabinogalactan, wherein said inducer is lactose.

Another aspect of the invention provides nesiritide synthetic cDNA chimera as set forth in Seq. ID NO:3.

Another aspect of the invention provides the yield of nesiritide chimeric protein in the range of 1 g/L to 8 g/L.

Yet another aspect of the invention provides a process for isolation and purification of nesiritide carried out by sequential steps comprising:
i isolating and solubilizing the inclusion bodies;
ii capturing the Nesiritide chimeric protein from the solubilized inclusion bodies;
iii digesting said protein of step ii by proteases;
iv precipitating the impurities to separate out Nesiritide in solution; and
v purifying the Nesiritide from the solution of step iv by chromatography.

Still another aspect of the invention provides nesiritide chimeric protein captured by expanded bed chromatography.

Yet another aspect of the invention provides proteolytic digestion of the chimeric protein by proteases selected from the group consisting of factor Xa, enterokinase, thrombin and trypsin wherein said protease is enterokinase.

Yet another aspect of the invention provides a process wherein impurities are precipitated out by adjusting pH to about 5.0 to 5.5.

Yet another aspect of the invention provides a process wherein nesiritide from the solution is purified by RP-HPLC or HIC, followed by desalting and buffer exchange.

Another aspect of the invention provides purified Nesiritide having mass of 3464 daltons and purity of ≧99%.

Another aspect of the invention provides purified Nesiritide having ED₅₀ in the range of 70 nM to 85 nM.

Another aspect of the invention provides a process for large scale purification of nesiritide comprising:
i solubilizing the inclusion bodies by treating with 8M Urea;
ii capturing the nesiritide chimeric protein using Iminodiacetic Acid (IDA) expanded bed chromatography;
iii digesting said protein of step ii by enterokinase;
iv precipitating the impurities at a pH of about 5.0 to 5.5 to separate out Nesiritide in solution;
v purifying the nesiritide from solution of step iv by HIC;
vi desalting the pure nesiritide of step v by ion exchange chromatography; and
vii performing buffer exchange on desalted pure Nesiritide of step vi.

Still another aspect of the invention claims the purity of nesiritide as ≧99.5%.

The cDNA nesiritide chimera as set forth in Seq. ID No.: 3 of the invention may be generated from one or more expression vector(s), each comprising regulatory elements operably linked and are also within the scope of the present invention.

The scope of the invention includes association of nucleic acid sequences provided by the invention with homologous or heterologous species expression control sequences, such as promoters, operators, regulators, and the like which allows for in vivo and in vitro transcription to the corresponding mRNA and subsequent translation to proteins and related poly- and oligo-peptides, in large quantities.

In a presently preferred expression system of the invention, nesiritide encoding sequences are operatively associated with a regulatory promoter sequence allowing for transcription and translation in prokaryotic cell system e.g. E. coli to provide recombinant nesiritide

The nucleotide sequence encoding cDNA nesiritide chimera as set forth in Seq. ID No.:3 may be inserted into an expression vector by conventional methods. Incorporation of these recombinant vectors into prokaryotic and eukaryotic host cells by standard transformation and transfection processes is also within the scope of the present invention and is expected to provide nesiritide in quantities greatly in excess of those obtainable from tissue sources. Appropriate host cells include E. coli, CHO and yeast systems.

Other aspects and advantages of the present invention will be apparent upon consideration of the following detailed description thereof which includes numerous illustrative examples of the practice of the invention, reference being made to the Sequence Listing wherein: SEQ ID NO: 3 provides the polynucleotide ORF sequence of the nesiritide chimera and SEQ ID NO:1 provides the polynucleotide sequence of the synthetic cDNA construct.

### BRIEF DESCRIPTION OF ACCOMPANYING DRAWINGS

The manner in which the objects and advantages of the invention may be obtained will appear more fully from the detailed description and accompanying drawings, which are as follows:

FIG. 1 shows the map of pET-RAZ-6-N vector which has the following components—

T7 promoter region: bases 5230-5246; Initiation ATG: bases 5317-5319; Polyhistidine tag: bases 5329-5346; β-galactosidase region: bases 5347-5490; Enterokinase recognition site: bases 5491-5505; Nesiritide: bases 5506-5607; T7 transcription termination region: bases 5721-5767; LacI ORF: bases 4845-3761; Amp^{r} ORF: bases 219-1069.

FIGS. 2a and 2b shows the comparison of wild type and Nesiritide of the present invention as regards RNA free energy and RNA secondary structure. Arrows in FIG. 2a are denoting the mutational changes made to the wild type sequences. FIG. 2A: Min. Energy: (−)31.25; Temperature: 37; GU is allowed. FIG. 2b: Min. Energy: (−)36.92; Temperature: 37; GU is allowed.

FIG. 3a shows fermentation profile for the Fed batch production of recombinant Nesiritide protein at 1 L scale.

3b shows fermentation profile for the Fed batch production of recombinant Nesiritide protein at 15 L scale.

FIG. 4 shows Coomassie stained SDS-PAGE profile of Nesiritide chimera purification using streamline chelating bead on expanded bed column. Lane 1: Crude Cell lysate in culture broth, Lane 2: Flow through of EBA column; Lane 3: Elution from EBA column.

FIG. 5 shows Coomassie stained SDS-PAGE of release of Nesiritide from chimeric protein by enterokinase digestion. Lane 1: EBA purified Nesiritide; Lane 2: Enterokinase digestion product of Nesiritide.

FIG. 6 shows the chromatographic profile of Nesiritide purification after EK digestion of chimeric protein using Source 30S cation exchanger column. The underlined peak fraction contains the peptide of interest.

FIG. 7 shows the final polishing of Nesiritide using reverse phase chromatographic technique on Source 15RPC. The underlined peak fraction contains the peptide of interest.

FIGS. 8a and 8b shows the final polishing of Nesiritide using hydrophobic interaction chromatography on Source phenyl matrix Inertsil ODS WPC18 column.

FIG. 9 shows comparison of the retention times of purified Nesiritide and reference standard Nesiritide on RP-HPLC. Upper line represents Nesiritide and lower line represents reference standard Nesiritide.

FIG. 10 shows the silver stained Tris-Tricine gel profile of purified Nesiritide versus reference standard Nesiritide. Lane 1 represents the size calibration standard, Lane 2 represents Nesiritide and Lane 3 represents reference standard Nesiritide.

FIG. 11 shows the tryptic digest map and comparison of the retention times of peptides generated by trypsin action in purified Nesiritide and reference standard Nesiritide. 1 represents Nesiritide and 2 represents reference standard Nesiritide.

FIG. 12 shows the ESI-MS analysis data of the purified Nesiritide and reference standard Nesiritide. Sample 1 represents reference standard Nesiritide (Bachem) and Sample 2 represents purified Nesiritide showing mass at 3464.01.

FIG. 13 shows cGMP induction of recombinant Nesiritide in PC-12 cells.

FIG. 14a shows comparison of bioactivity of Nesiritide samples with standards. In all five batches of Nesiritide of the present invention, the cGMP induction in PC-12 cells the Nesiritide concentration (nM) is directly proportional to the concentration of Nesiritide used in culture. Culture format: 24-well plate; Cells used: PC-12 cells (Rat adrenal pheochromocytoma); no. of cells plated: 1.0×106 cells per well; Parameter measured: cGMP from cell lysate; Method used: Competitive Inhibition Assay (Acetylated format); Kit used: Correlate-EIA cGMP kit Assay Design Inc., USA.

FIG. 14b shows immunoreactivity of Nesiritide samples and standards by direct ELISA. The antigen concentration used are 10 μg/ml, 5 μg/ml, 1 μg/ml, 0.5 μg/ml, 0.1 μg/ml diluted in coating buffer pH 9.6 by adding 100 μl/well. Primary antibody titre: Anti-BNP antibody 1:1000; Secondary antibody titre: Anti-rabbit HRPO 1:40,000. All the five batches of Nesiritide are comparable with that of standards with sensitivity of detection by ELISA at 10 ng/well.

### DETAILED DESCRIPTION OF THE INVENTION

One embodiment of the present invention provides an improved process for synthesis of Nesiritide as set forth in Seq. ID NO:2 comprising:
ii. preparing a synthetic cDNA construct as set forth in Seq. ID No. 1 encoding ORF of Nesiritide polynucleotide by iterative optimization of RNA transcript free energy operably in the range of (−)30 Kilo calories to (−)300 Kilo calories per mole;
iii. fusing said polynucleotide of step i) with a fusion partner consisting essentially of 41 amino acids from the N-terminal region of E. coli β-galactosidase and an affinity handle operably linked to regulatory elements cloned in an expression vector;
iv. expressing the synthetic cDNA chimera as set forth in Seq. ID No. 3 in a host by culturing the host cells in the growth medium under appropriate conditions to yield Nesiritide chimeric protein; and
iv isolating and purifying the Nesiritide obtained from step iii.

Another embodiment of the invention provides a synthetic cDNA construct as set forth in Seq. ID NO:1 prepared by sequential PCR cloning technique, preferably by primer driven codon optimization of the gene.

Still another embodiment of the invention provides gene-specific primers selected from Seq. ID NO:4 to Seq. ID NO:21.

Still another embodiment of the invention provides an expression vector consisting of synthetic cDNA chimeric sequence as set forth in Seq. ID NO. 3 wherein the

expression vector is pET-RAZ-6-N having sequence as set forth in Seq. ID NO:22 with cDNA chimeric sequence from 5315 bp to 5607 bp.

Still another embodiment of the invention provides the affinity handle to be selected from maltose-binding protein, poly Histidine and staphylococcal protein wherein said affinity handle is polyhistidine.

Yet another embodiment of the invention provides the host as E. coli wherein E. coli harbours the expression vector pET-RAZ-6-N as set forth in Seq. ID NO:22 having cDNA chimeric sequence from 5315 bp to 5607 bp, said vector comprising synthetic cDNA chimeric sequence as set forth in Seq. ID NO:3.

A further embodiment of the invention is directed to the expression of synthetic cDNA chimera as set forth in Seq. ID No. 3 which encodes nesiritide under the control of a promoter, selected from a group consisting of ara_{BAD}, trp, T7, lac, pho, and trc, wherein the promoter is T7.

Still further embodiment of the invention provides growth medium for culturing host cells comprising an inducer selected from a group consisting of Isopropyl-beta-D-thiogalactopyranoside (IPTG), lactose, maltose, arabinose and arabino galactan, wherein said inducer is lactose.

Another embodiment of the invention provides nesiritide synthetic cDNA chimera as set forth in Seq. ID NO:3.

Another embodiment of the invention provides the yield of nesiritide chimeric protein is in the range of 1 g/L to 8 g/L.

Yet another embodiment of the invention provides a process for isolation and purification of nesiritide carried out by sequential steps comprising:
i isolating and solubilizing the inclusion bodies;
ii capturing the Nesiritide chimeric protein from the solubilized inclusion bodies;
iii digesting said protein of step ii by proteases;
iv precipitating the impurities to separate out Nesiritide in solution; and
v purifying the Nesiritide from the solution of step iv by chromatography.

Still another embodiment of the invention provides nesiritide chimeric protein captured by expanded bed chromatography.

Yet another embodiment of the invention provides proteolytic digestion of chimeric protein by proteases selected from the group consisting of factor Xa, enterokinase, thrombin and trypsin wherein said protease is enterokinase.

Yet another embodiment of the invention provides a process wherein impurities are precipitated out by adjusting pH to about 5.0 to 5.5.

Yet another embodiment of the invention provides a process wherein nesiritide from the solution is purified by RP-HPLC or HIC, followed by desalting and buffer exchange.

Another embodiment of the invention provides purified Nesiritide having mass of 3464 daltons and purity of ≧99%.

Another embodiment of the invention provides purified Nesiritide having ED₅₀ in the range of 70 nM to 85 nM.

Another embodiment of the invention provides a process for large scale purification of nesiritide comprising:
i solubilizing the inclusion bodies by treating with 8M Urea;
ii capturing the nesiritide chimeric protein using Iminodiacetic Acid (IDA) expanded bed chromatography;
iii digesting said protein of step ii by enterokinase;
iv precipitating the impurities at a pH of about 5.0 to 5.5 to separate out Nesiritide in solution;
v purifying the nesiritide from solution of step iv by HIC;
vi desalting the pure nesiritide of step v by ion exchange chromatography; and
vii performing buffer exchange on desalted pure Nesiritide of step vi.

Still another embodiment of the invention provides the purity of nesiritide as ≧99.5%.

The term “cDNA” or complementary DNA as used herein refers to synthetic DNA reverse transcribed from a specific RNA through the action of the enzyme reverse transcriptase. “Synthetic cDNA construct” as used herein refers to DNA construct prepared synthetically by sequential PCR cloning technique e.g. by primer driven codon optimization of the gene.

The term “ORF” or open reading frame as used herein refers to a portion of organism's genome which contains a sequence of bases potentially encoding a protein. In this state, it is known that ORFs are located between the initiation codon and termination codons. Herein, ORF refers to synthetic polynucleotide encoding nesiritide without any introns.

As used herein, the term “iterative optimization of RNA transcript free energy” refers to evaluation of total RNA free energy by changing single nucleotide in the ORF without changing the coded amino acid for improving the RNA stability for better expression of the gene of interest. The secondary structure of the RNA can be predicted and overall free energy value can be assigned to the RNA secondary structure. It is thus possible to design a synthetic RNA which is stable.

By “RNA transcript free energy” is meant amount of energy required in kilo calories per mole by translational machinery to linearise the RNA.

By “operably linked” is meant that transcriptional and translational regulatory polynucleotides are positioned relative to a polypeptide-encoding polynucleotide in such a manner that the polynucleotide is transcribed and the polypeptide is translated.

By “regulatory elements” is meant a biological functionality comprising of origin of replication, a promoter, an operator, a termination sequence, an initiation sequence and a ribosome binding site.

By “promoter” is meant a DNA sequence which is recognized by an RNA polymerase and which directs initiation of transcription at a nearby downstream sequence. As used herein “promoter” refers to viral or bacterial transcriptional control sequences.

By “expression vector” is meant any autonomous genetic element capable of directing the synthesis of a protein encoded by the vector. Such expression vectors are known by practitioners in the art.

By “synthetic cDNA chimera” is meant synthetic cDNA encoding protein of interest fused with a heterologous fusion partner native to the host for enhanced expression of protein of interest and optionally an affinity handle facilitating purification.

By “growth medium” is meant any medium used for fermentation comprising water, oxygen, energy source, carbon source, nitrogen source and micronutrients for growth. In addition the growth medium may also be supplemented with trace elements, antifoaming agents, buffers, growth factors, precursors, inhibitors, inducers, and chelaters to avoid precipitation of metal ions.

By “under appropriate conditions” is meant culturing the host cells under appropriate fermentation parameters such as defined aeration rate, temperature, dissolved oxygen (DO), stirrer speed, induction period, feed rate, pH maintenance, total fermentation cycle, and harvest period for the cultured cells.

By “primer” is meant an oligonucleotide which, when paired with a strand of DNA, is capable of initiating the synthesis of a extension product in the presence of a suitable polymerizing agent. The primer is preferably single stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues.

By “gene specific primers” is meant primers sufficiently complementary to hybridize with a target polynucleotide for the synthesis of the extension product of the primer which is complimentary to the target polynucleotide.

By “large scale purification” is meant any down stream purification process for industrial production of any protein of interest. E.g. purification at 15 L scale.

By “ED₅₀ value” is meant a dose at which 50% effect is seen, expressed in nano moles.

It was experimentally verified for the first time that the expression of nesiritide can be improved by competent construction of a synthetic DNA construct encoding nesiritide gene. The present invention circumvents the difficult step of isolation of mRNA from genomic sources and directly gives the polynucleotide ORF sequence of nesiritide. The inherent advantages of the synthetic construction of the gene are already known in the prior art. To list a few, synthetic construction offers the following advantages: i) directly gives the desired sequence. The coding sequence and the non-coding sequences can be designed at will for prokaryotic expression, restriction sites can be removed or built in, introns added or deleted; ii) can bypass the often difficult step of isolating the relevant mRNA or genomic DNA; and iii) simplifying the modification of the gene and its product protein by lengthening or shortening the coding region, or by changing specific codons and the corresponding amino acids. In the preferred embodiment of the present invention, a synthetic DNA encoding the 32 amino acids of nesiritide is constructed using recombinant techniques by RNA free energy optimization and codon wobble resulting in improved RNA stability for the said RNA transcript.

Synthetic DNA fragments can be synthesized as follows: i) select an unique protein sequence, ii) reverse translate to determine complementary DNA sequence, iii) optimize codons for bacterial or yeast expression, and iv) introduce and/or remove specific restriction sites. Codon optimisation often times involves increased unfavorable RNA free energy with >10 RNA: RNA base pairing with high GC content.

Mutagenesis of a codon without changing the amino acid sequence with simultaneous reduction of free energy from nearest neighbour complimentarity in the transcript is unique to this invention.

The iterative process of codon optimization and subsequent convergence of minimum RNA free energy is yet another novel feature of this invention.

The energy optimization with codon wobble are a set of steps that are aimed to minimize free energy from −300 Kcal/mol to about −30 Kcal/mol for the said RNA transcript from which the DNA ORF is read out. Thus, minimization of total free energy by wobble perturbation is still another novel feature of the present invention.

A native gene will generally tend to exhibit the codon usage or preference of the particular organism from which it is derived. Expression of eukaryotic gene products in prokaryotes is sometimes limited by the presence of codons that are infrequently used in E. coli. It is commonly considered that rare codons cause pausing of the ribosome. Pausing of the ribosome can lead to failure to complete the nascent polypeptide chain and an uncoupling of transcription and translation. Additionally, pausing of the ribosome is thought to expose the 3′ end of the mRNA to cellular ribonucleases. By replacing the codons in a human protein-coding sequence with codons with a higher frequency in E. coli, it is possible to increase percent expression of the human protein in E. coli under defined conditions. Expression of such genes can be enhanced by systematic substitution of the endogenous codons with codons over represented in highly expressed prokaryotic genes. U.S. Pat. No. 6,114,148 features a synthetic gene that encodes a protein normally expressed in a mammalian cell wherein a non-preferred codon in the natural gene encoding the protein has been replaced by a preferred codon encoding the same amino acid. Furthermore, there are species specific differences with codons that are preferred, or less preferred among species of a genus (Narun et al., 2001).

In the present invention, Table 1 depicts the usage of highly expressed codons in E. coli incorporated in the synthetic cDNA construct encoding the polynucleotide ORF as set forth in Seq. ID No. 1 that are aimed to minimize free energy to about—30 Kcal/mol for the said RNA transcript as against the human genomic sequence codons (depicted in U.S. Pat. No. 5,114,923 patent) by the innovator. A synthetic gene coding for the 32 amino acids was designed in two steps. First, amino acid codons found in the human genomic sequence for recombinant nesiritide (U.S. Pat. No. 5,114,923) were compared to amino acid codons found in highly expressed E. coli genes (Current Protocols in Molecular Biology, Supplement 33, Pg.: A.IC.3, John Wiley & Sons Inc., 1994). Only two of 6 Ser codons were of the type found in highly expressed E. coli genes. Similarly, none of the four Arg codons were found to be of preferred sequence, CGC. Overall, only 47% of the codons found in the human genomic sequence for recombinant nesiritide (U.S. Pat. No. 5,114,923) were those favoured for high level expression in E. coli.

For amino acids such as Val, Met, Leu, His and Cys where no clear codon preference has been demonstrated, the original nucleotide triplet from human genomic sequence for recombinant nesiritide are retained. A preferred embodiment of the present invention is thus assembling a synthetic recombinant nesiritide gene in which the proportion of E. coli preferred codons was increased from 47% to 94%. The present invention is aimed for improvement in yield which is a direct result of codon usage bias.

TABLE 1
Comparison of codons used in the synthesis of Nesiritide

| | Codon in | | | |
|---|---|---|---|---|
| Amino | Human BNP | Frequency | Codon used in | Frequency of |
| acid | Sequence | of usage | this invention | usage |
| Ser | AGC | 16.6 | AGC | 16.6 |
| Pro | CCC | 5.8 | CCG | 23.7 |
| Lys | AAG | 14.2 | AAA | 33.9 |
| Met | ATG | 25.2 | ATG | 25.2 |
| Val | GTG | 23.8 | GTG | 23.8 |
| Gln | CAA | 12.5 | CAG | 29.4 |
| Gly | GGG | 9.4 | GGC | 34.9 |
| Ser | TCT | 9.6 | AGC | 16.6 |
| Gly | GGC | 34.9 | GGC | 34.9 |
| Cys | TGC | 6.8 | TGC | 6.8 |
| Phe | TTT | 17.1 | TTC | 18.9 |
| Gly | GGG | 9.4 | GGC | 34.9 |
| Arg | AGG | 1.9 | CGC | 23.5 |
| Lys | AAG | 14.2 | AAA | 33.9 |
| Met | ATG | 25.2 | ATG | 25.2 |
| Asp | GAC | 25.1 | GAT | 30.9 |
| Arg | CGG | 5.6 | CGC | 23.5 |
| Ile | ATC | 28.0 | ATC | 28.0 |
| Ser | AGC | 16.6 | AGC | 16.6 |
| Ser | TCC | 10.7 | AGC | 16.6 |
| Ser | TCC | 10.7 | AGC | 16.6 |
| Ser | AGT | 7.0 | TCA | 6.4 |
| Gly | GGC | 34.9 | GGC | 34.9 |
| Leu | CTG | 51.5 | CTG | 51.5 |
| Gly | GGC | 34.9 | GGC | 34.9 |
| Cys | TGC | 6.8 | TGC | 6.8 |
| Lys | AAA | 33.9 | AAA | 33.9 |
| Val | GTG | 23.8 | GTT | 18.0 |
| Leu | CTG | 51.5 | CTG | 51.5 |
| Arg | AGG | 1.9 | CGC | 23.5 |
| Arg | CGG | 5.6 | CGT | 21.4 |
| His | CAT | 11.1 | CAT | 11.1 |

It is well known in the art that RNA plays many roles in biological processes and RNA structure can improve the understanding of its biological functions. RNA secondary structures can be deduced from a phylogenetic comprison of many related sequences or from calculation of free energies based on thermodynamic measurements of model oligonucleotides for stable secondary structures. The present invention features the replacement of the majority of the rare codons wherein the rare arginine codons (AGG and CGG) are replaced by highly expressed codon (CGC) and rare proline codon CCC by CCG. Altering the nucleotide sequence have altered the potential secondary structure and/or stability of mRNA and level of gene expression. The wobble perturbation aids in stabilizing the RNA from which the nesiritide ORF is read by minimization of free energy from −300 Kcal/mole to −30 Kcal/mole wherein the expression is enhanced yielding 1.0 g/L to 8.0 g/L of the peptide of interest. Table 2 shows the effect of codon wobble on RNA transcript free energy.

TABLE 2
Effect of codon wobble on transcriptional free energies

| | Transcriptional Free energy* |
|---|---|
| RNA stretch | (Kcal/mol) |
| Nesiritide | −31.25 at 37° C., GU allowed |
| Innovator's Nesiritide | −36.92 at 37° C., GU allowed |
| Nesiritide as a fusion with a | −109.36 at 37° C., GU |
| portion of β- | allowed |
| Galactosidase(theoretical) | |
| Innovator's Nesiritide as a fusion | −115.23 at 37° C., GU |
| with a portion of β-Galactosidase | allowed |

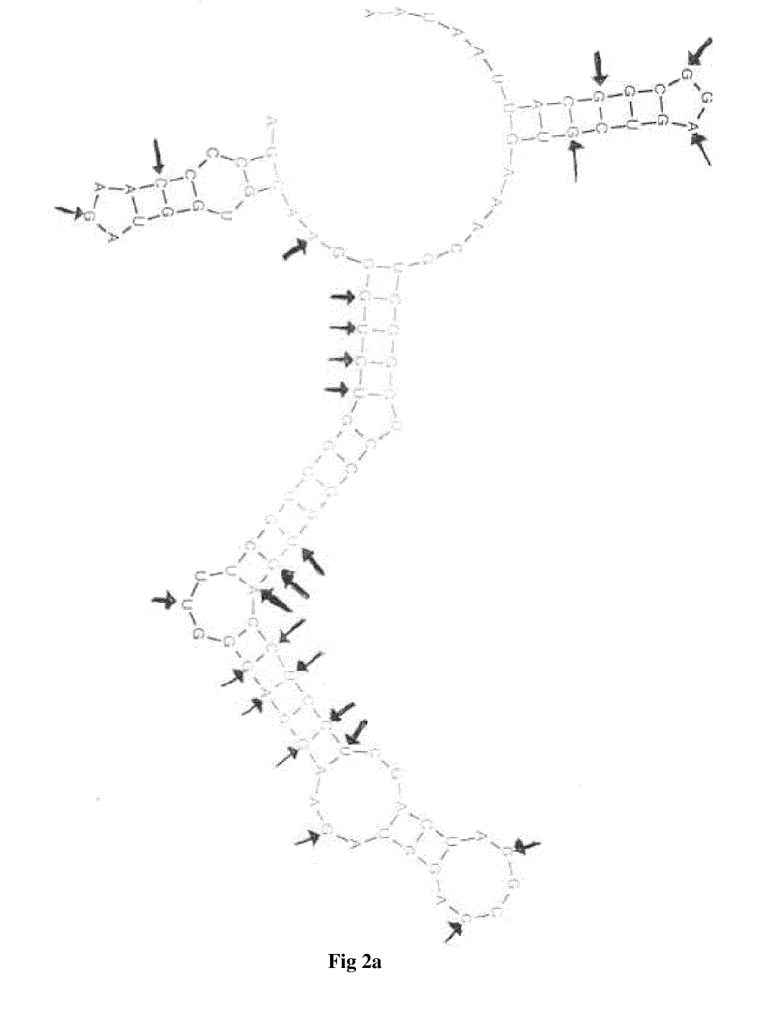

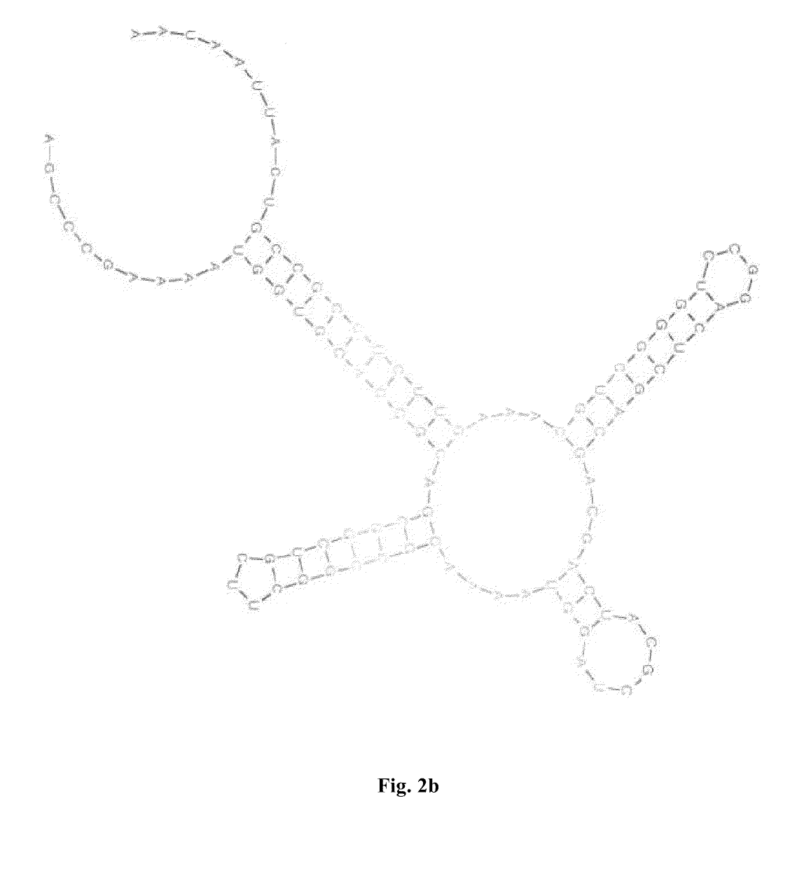

*Note that a single muation in ORF leads to major changes in free energies. In the present invention, 27 nucleotides in wild type Nesiritide were changed without much impact on RNA free energies. The mutations in nesiritide ORF were chosen such that the free energy of the RNA secondary structure was not altered substantially (FIG. 2a and 2b).

Direct expression of small polypeptides in E. Coli is unsuccessful in many cases since short peptides are often subject to degradation by proteases in the host cells which leads to significant decrease in yields of product. It also creates more difficulties for separating target peptides from their degraded fragments. Expression levels can be improved by linking the eukaryotic gene with a bacterial gene and producing a fused protein product. Fusion proteins can be expressed to levels of up to 26% of total cell protein, but if the bacterial gene constitutes a large proportion of the fusion, then the amount of eukaryotic product will be small.

The present invention provides a truncated E. Coli β-galactosidase fragment as a fusion partner resulting in a stable fusion protein. A novel cDNA nesiritide chimera as set forth in Seq. ID No.:3 is constructed encoding the fusion protein wherein the fusion partners are 41, 64 and 86 amino acids residues from N-terminal region of E. Coli β-galactosidase. The expression vector harboring the cloned polynucleotides are inserted in E. coli capable of replicating the said expression vector and expressing the chimeric protein in the form of inclusion bodies. Table 3 illustrates a comparative analysis showing the expression levels of the chimeric proteins. As a direct contribution of the leader sequence, it was surprisingly found that the fusion partner with 41 amino acid residues from N-terminal region of E. Coli β-galactosidase exhibits higher expression.

TABLE 3
Comparison of expression of nesiritide with different fusion partners

| Sr. | | |
|---|---|---|
| no | Fusion partner | % expression* |
| 1. | 6X His | 0.5-1 |
| 2. | 6X His-86 A.As of β-Galactosidase | 0.5-1 |
| 3. | 6X His-64 A.As of β-Galactosidase | 0.5-1 |
| 4. | 6X His-41 A.As of β-Galactosidase | 25-30 |
| 5. | 6X His-human Growth Hormone- | 0.5-1 |
| | 41 A.As of β-Galactosidase | |

*As calculated from densitrometric scan of SDS-PAGE gels

The present invention also aims at codon optimized synthetic nesiritide gene as a novel fusion protein wherein the preferred fusion partner is 41 amino acids from N-terminal region of E. coli β-galactosidase highly expressed in the host cells with an affinity tag. This not only elevates the expression levels of the chimeric protein and

thus the yield of the target peptide but also facilitates purification of the chimeric protein.

Post fermentation methods, the inclusion bodies are recovered from the host cell. The cells are disrupted by known techniques, e.g., sonication or high pressure homogenization at controlled temperatures. The crude cell lysate is then centrifuged at low speeds to pellet the inclusion bodies that have higher density than the other cellular impurities. The inclusion bodies are then solubilised using high concentration of chaotropic agent e.g., 8 M urea or 6 M guanidine hydrochloride and further purified by chromatographic or other techniques.

An alternative to the above method, as incorporated in the present invention, involves direct solubilisation of proteins in the crude cell lysate (rather than inclusion body stabilisation) followed by chromatographic purification. This is achieved by addition of Urea to 8M or Guanidine Hydrochloride to 6M final concentration and stirring the solution for sufficient time for the inclusion body proteins to solubilize. The crude lysate containing the solubilized chimeric fusion protein can then be directly purified on affinity or ion-exchange media using expanded bed chromatographic procedure as well as combination of standard chromatographic techniques.

Once the tagged chimeric protein is purified, the peptide needs to be released from the fusion and subsequently purified. Release of peptide can be achieved in a number of ways—enzymatic or chemical. The present invention is directed to use of enzymatic cleavage to cleave the target peptide from its fusion partner. The process involves use of an enzyme or chemical that cleaves specifically between the fusion tag and the peptide, releasing the peptide with intact N-terminal amino acid as that found in the natural peptide in vivo. For those skilled in the art, the use of enzymes like recombinant Enterokinase light chain, Thrombin and Factor Xa that have specific cutting properties to obtain protein/peptide with intact N-terminus from fusions is well known. The use of chemicals is limited as the conditions used are often harsh and extensive damage to the protein/peptide may occur. Dilute mineral acids have been used to cleave peptides from fusions. However such reactions are frequently non-specific and difficult to control. Hence, use of enterokinase for specifically cleaving nesiritide with intact N-terminus is an unique feature of the present invention.

The present invention is also directed to the use of pH precipitation which is particularly useful for purification of peptides. The present invention takes advantage of relatively high pI (>10) of nesiritide, as compared to the fusion tag as well as the uncleaved chimeric protein (pI˜6.0). Hence after cleavage reaction, the fusion proteins can be easily pH precipitated leaving practically all the peptide in solution. The main advantage of the precipitation is the relative ease of use. An additional advantage of the technique is that very large process volumes can be handled. In addition, precipitating agents can be chosen that provide a more stable product than found in the soluble form. This economic benefit results in a cost effective, commercially feasible and less energy intensive process.

The present invention uses of PCR driven techniques for generation of nesiritide ORF. Amplification was done using specially designed primers Seq ID No 4 & 5, a suitable expression vector (pBAD/His, pcDNA3.1 Myc/His, pET series, pRA, etc) as template and DNA polymerase enzyme under following amplification conditions—(25-35 cycles of denaturation at 95° C. for 1 min, annealing at 54-63° C. for 2 min and extension at 72° C. for 1 min). The amplified fragment containing sequences coding for the first n (n=1-15) amino acids of the desired product and an expression vector such as pBAD/His, pET series, pRA, pcDNA3.1 Myc/His etc were then digested with restriction enzymes (Age I, Sal I, Nco I, Pvu I), in 1× buffer at 25° C.-55° C. overnight and purified. (The digested DNA was run on an agarose gel (0.8-1.5%) containing ethidium bromide and the desired fragments were cut out from the gel. The agarose was dissolved in sodium iodide solution at 50° C.-60° C. and the DNA was purified using Qiaquick PCR purification kit (Qiagen). DNA sample to be purified was mixed with 3-5 volumes of Buffer PB provided in the kit and applied to a Qiaquick column. This was spun for 30-60 secs at 14K rpm and the flow through was discarded. The membrane was washed with the wash buffer provided in the kit and the DNA was eluted with nuclease free distilled water). This was followed by ligation of the digested products to create an intermediate vector having n (n=1-15) amino acids of the desired product.

Ligation was done using T4 DNA Ligase enzyme in ligation buffer containing 40 mM-50 mM Tris-HCl, 10 mM MgCl₂, 1 mM-10 mM DTT and 0.5 mM-1 mM ATP (pH 7.6-7.8) at R.T/37° C. for 20 mins-2 hrs followed by an overnight incubation at 4° C.-12° C. The intermediate vector was used as a template for amplification with specific primers (Seq ID No 4 and 6) as described above. The amplified fragment containing sequences coding for the next m (m=1−15) amino acids of the desired product and a procaryotic expression vector such as the inhouse procaryotic expression vector pRA were then digested with restriction enzymes in 1× buffer at 25° C.-55° C. overnight, purified using the Qiaquick PCR purification kit (Qiagen) followed by ligation of the digested products to create a second intermediate vector (having the first p (p=2−30) amino acids of the desired product) which was used as template for another round of amplification. The above steps were repeated eight more times and the final PCR product was purified and then digested with restriction enzymes in respective 1× buffer at 25-55° C. This was then ligated to the digested expression vector such as pBAD/His, pET series, the inhouse procaryotic expression vector pRA, pcDNA3.1 Myc/His etc to create the desired expression vector of this invention which has an affinity peptide (6×His) linked to a EK protease cut site followed by the generated DNA sequence of interest (pRA-N containing 32 amino acids of the desired product).

The PCR primers were generated based on a sequence that passed energy minimization of the transcript. Firstly, the wild type ORF was juxtaposed with the promoter to get the full RNA transcript. The secondary structure of the RNA transcript was analyzed. The free energies and sequences of RNA:RNA hybrid was tabulated. Iterative mutations in the third position of the codon (wobble) was attempted to generate a convergent series of free energies (−30 to −300 Kcal/mole) (FIGS. 2a & 2b). The optimised free energy of the transcript was taken into consideration during the primer-derived mutational insertion and creation of a synthetic ORF on a plasmid vector which is one of the focus of this invention. Using the above methodology, pRA-N vector (which has the affinity peptide 6× His linked to EK site which is linked to gene coding for the desired product controlled by arabinose inducible promoter), pRAZ-6-N (which has the affinity peptide 6×His linked to 123 bp of N-terminal region of E. coli β-galactosidase gene followed by EK site and the desired product controlled by arabinose inducible promoter) and pET-N (which has the affinity peptide 6×His linked to 123 bp of N-terminal region of LacZ gene followed by EK site and the desired product controlled by T7 promoter) were constructed. All of the above constructs comprise the following: Promoter, affinity handle (6×His), an optional 123 bp of N-terminal region of E. coli β-galactosidase gene, EK protease cut site, gene coding for the desired product, transcription termination region, gene for ampicillin resistance, origin of replication and other coli-based sequences and may be in any interchangeable sequence.

The culture (E. coli strain transformed with a plasmid containing DNA fragment encoding nesiritide) from glycerol stock was streaked on a 2× Yeast Tryptone plate which was incubated at 37° C. for 16-24 hrs. Single colony from the plate was inoculated in 10 ml of 2× Yeast Tryptone liquid medium and grown at 37° C. on rotary shaker (200 to 220 rpm) for 16 hrs. The grown culture was transferred to 100 mL of basal fermentation medium (seed medium) in a 500 mL conical flask and grown at 37° C. on a rotary shaker (200 to 220 rpm) for 8 hrs. 100 ml of seed culture was transferred to 900 ml of fermentation medium in 2 L jar fermentor. Inducer solution was added between 12 to 20 hours of growth. Antifoam solution was fed as and when excess foaming was observed. At various time-points during the fermentation run, aliquots were withdrawn from the fermentor and the OD of the sample was determined by spectrophotometry and percent expression and yield of Nesiritide was determined.

At the end of the fermentation, the culture broth was harvested from the fermentor. This broth was then directly subjected to multiple cycles of high pressure homogenisation at 850-900 bar using Panda 2K homogeniser from Niro Soavi. The crude cell lysate was then buffered using sodium phosphate and solid urea crystals added to give 6-8 M final concentration. This was stirred vigorously for 12-16 hours at ambient temperatures to ensure maximal dissolution of the inclusion bodies containing the His-tagged fusion protein. This solution was then diluted with water to bring down the chaotrope concentration to 2-4 M and the diluted solution pumped into a Streamline column from Amersham containing Chelating Sepharose beads (Amersham), charged with either Cu²⁺ or Ni²⁺ After loading was complete, the cellular impurities and unbound proteins were removed by washing with buffer containing 20-50 mM imidazole, preferably 30-40 mM. The bound proteins were then eluted from the column with buffer containing 100-500 mM imidazole, preferably 200-250 mM final concentration. However, other means of elution could also be used, preferably using Histidine at similar concentrations. Elution was monitored by UV absorption at 280 nm and the major peak fraction collected and then rapidly desalted on a Sephadex G-25 column to remove salt and imidazole. Protein concentration in the peak fraction was determined using Bradford's dye binding method and the concentration adjusted to give 4-8 mg/ml, more preferably 5-6 mg/ml final concentration. This solution was then rapidly chilled to below 10° C., more preferably between 5-6° C. and kept under gentle stirring.

Recombinant Enterokinase was then added at a concentration of 1-10 unit per 20-100 ug protein, more preferably at a ratio of 1-5 unit per 50-75 ug fusion protein and the solution gently stirred for 4-16 hours. The release of peptide was monitored at 214 nm using reverse phase chromatography and after optimal release of the peptide was obtained, the solution was removed from the cold and rapidly allowed to attain ambient temperatures.

The undigested fusion protein, the fusion tag and other contaminating proteins can be removed by novel use of pH adjustment of the solution to below 6, more preferably to between 4.5 to 5.0 with dilute acid. The precipitate can then be removed by centrifugation at low speeds or by microfiltration using tangential flow filtration. The clarified supernatant or filtrate containing maximal amount of the released peptide was further purified to obtain the pure peptide. However, precipitation as a purification method in the present invention for nesiritide was sub-optimal for the removal of closely related peptide impurities and also from large-scale purification point of view. Thus, the process of the instant invention, preferably uses a cation exchanger to specifically capture the peptide and peptide-related impurities and then resolve them using a simultaneous gradient of salt and solvent. Source 30S, a polymeric cation exchanger from Amersham was the preferred choice of the matrix. However other commercially available matrix containing the sulfopropyl group on polymeric beads can be used to obtain similar results.

The enzyme cleavage solution was clarified by microfiltration, preferably using 0.22 u filter and the clarified solution was then pumped into ion-exchange column, previously equilibrated with low concentration of buffer, preferably 10-100 mM sodium phosphate at pH 6-8 containing 2-6 M urea. After loading was complete the unbound proteins were washed with the above buffer followed by the same buffer without urea. The column was then washed with low concentration buffer containing 2-10% solvent e.g., acetonitrile, methanol, ethanol or isopropyl alcohol. The bound peptides were then eluted from the column using a novel gradient of above buffer and a buffer containing 20-50% acetonitrile and high salt, preferably 25% acetonitrile and 400-600 mM salt, preferably NaCl in the field of this invention. Elution was monitored at 214-220 nm.

The peptide fraction was further purified, as desired, by additional chromatographic steps, most preferably reverse phase chromatography on Silica or Polymeric matrices. In the present invention, Source 15RPC, a 15 micron polymeric reverse phase media from Amersham, was used to polish the peptide to purity that is suitable for therapeutic use. Sodium phosphate at low concentrations having pH between 2-3 was used as buffer A and elution was effected with a gradient of 70-90% Acetonitrile in water which is further novel feature of the present invention. The peak of interest containing high purity B-type natriuretic peptide was collected and diluted with water. This was then loaded on a small column containing reverse phase media and equilibrated with dilute acid solution, preferably 0.01-0.1% acetic acid in water. After complete sample load the column was washed extensively with the same buffer and the bound peptide eluted with high concentration of Acetonitrile in water. The peak fraction was collected and lyophillised to obtain pure acetate form of the peptide suitable for therapeutic use.

Alternatively for large scale purification of nesiritide, e.g. at 15 L scale, hydrophobic interaction chromatography was used which has several advantages well known in the art. RP-HPLC for polishing nesiritide to purity involves use of organic solvents driving investments in terms of a flameproof facility for safe handling and disposal of solvents like acetonitrile and methanol. Moreover, RP-HPLC is a energy intensive process operating at high pressures as against a low pressure operation of HIC.

The nesiritide eluate from cation exchange column is directly loaded onto a column packed with a hydrophobic matrix having phenyl functional groups attached to a resin derived from cellulose, agarose, dextran, synthetic polymers or their derivatives. The column was equilibrated at a pH of about 7.0 in a suitable buffer containing 0.05M sodium phosphate and 2M ammonium sulphate. The bound nesiritide was eluted using linear gradient system. The nesiritide protein after this step was subjected to second HIC step after 1:1 dilution with a buffer containing 0.05M sodium phosphate and 3M ammonium sulphate. The column was equilibrated at a pH of 8.0 with a buffer containing 0.01M sodium acetate of pH 5.0. The peak fraction after this step was buffer exchanged with final storage buffer and lyophilized without loss of activity.

### GENERAL METHODOLOGY

The procaryotic expression vector pRA was used as a template for a polymerase chain reaction (PCR) where the following reagents were added in a sterile tube —

| | |
|---|---|
| DNA | 20-200 ng |
| 10X PCR Buffer - MgCl_{2*} | Final 1X |
| Primer-1(25uM) | Final 5-50pM |
| Primer-2 (25uM) | Final 5-50pM |
| 25 mM MgCl_{2**} | Final 1-2 mM |
| Taq/Pfu Polymerase | Final 1-2.5U |
| Nuclease free water | To make up volume |

*If Pfu polymerase enzyme is used, use 10× PCR buffer containing MgSO₄.
**If Pfu polymerase enzyme is used, MgCl₂ is not added.

The ingredients are mixed as they are added along. After the addition of the last reagent, the mixture is pulse centrifuged and then heated at 90-95° C. for 30 sec-2 min in the thermal cycler (optional) and then paused. 2 mM dNTP (preheated for 1 min at 90° C.—optional) is then added so that the final concentration is 10-300 uMoles. After adding dNTP's, the solution is mixed well by pipetting up and down, loading on the thermocycler rack and a preset programme in the PCR machine is started.

Amplification was done using specially designed primers (Seq ID No 4-15), purified template and Taq/Pfu polymerase enzyme (1-2.5 units/ul, MBI) under different cycling conditions (eg. 25-35 cycles of denaturation at 95° C. for 1-2 min, annealing at 45° C.-70° C. for 1-2 min and extension at 72° C. for 1-2 min. For analysis of the PCR products, 5-10 ul of sample was mixed with 1-10 ul of 1XLoading Dye and run on 0.8%-1.5% Agarose Gel as required.

The procaryotic expression vector pRA and PCR product containing the first 4 amino acids of the desired product were then digested with restriction enzymes (such as Xho I, Age I, Ase I etc.) in 1× buffer at 25° C.-55° C. overnight, purified using the Qiaquick PCR purification kit (Qiagen) followed by ligation of the digested DNAs. The digested pRA vector was run on an agarose gel (0.8-1.5%) containing ethidium bromide and the desired fragments were cut out from the gel. The agarose was dissolved in sodium iodide solution at 50° C.-60° C. and the DNA was purified using Qiaquick PCR purification kit (Qiagen) as described earlier.

Ligation was done as mentioned earlier. The purified ligated product was used as a template for PCR using specifically designed primers as mentioned above. The primers were designed on a synthetically generated nesiritide ORF. This PCR product was purified followed by digestions with restriction enzymes in respective 1× buffers at 25° C.-55° C. overnight, purification of the digested DNAs and ligations to create intermediate vectors. The above steps were repeated 9 times, each time adding sequences coding for more amino acids of the desired product, to create the construct pRA-N.

To create pRAZ-6-N, the construct expressing nesiritide ORF as a fusion with 41 amino acids of N-terminal region of β-Galactosidase protein, amplification was done using specially designed primers (Seq ID No 16 and 17), an in-house construct of GCSF (with the same fusion partner—pRAZ-5-GCSF) as the template and Taq/Pfu polymerase enzyme (1-2.5 units/ul, MBI) under different cycling conditions (eg. 25-35 cycles of denaturation at 95° C. for 1-2 min, annealing at 45° C.-70° C. for 1-2 min and extension at 72° C. for 1-2 min. This PCR product was purified and the purified DNA along with pRA-N vector were digested with restriction enzymes in respective 1× buffers at 25° C.-55° C. overnight followed by purification of the digested DNAs and ligations to create pRAZ-6-N. This construct has the affinity peptide (6×His) linked to 123 bp of N-terminal region of E. coli β-galactosidase gene followed by an Enterokinase site which is linked to ORF of the desired product controlled by arabinose inducible promoter and operably linked to the vector either as a single or a dual cassette. pRAZ-6-N was used as the template in two amplification reactions with specially designed primers (Seq ID No 18 to 21) and Taq/Pfu polymerase enzyme (1-2.5 units/ul, MBI) under different cycling conditions (eg. 25-35 cycles of denaturation at 95° C. for 1-2 min, annealing at 45° C.-70° C. for 1-2 min and extension at 72° C. for 1-2 min. The amplified products were purified, digested with restriction enzymes in respective 1× buffers at 25° C.-55° C. overnight followed by purification of the digested DNAs and ligations. The ligated product was purified and ligated to the fragment generated by digestion of pET19b vector with restriction enzymes to create pET-RAZ-6-N. This construct has the affinity peptide (6×His) linked to 123 bp of N-terminal region of E. coli β-galactosidase gene followed by an EK site which is linked to ORF of the desired product controlled by T7 promoter.

It should be understood that the following examples described herein are for illustrative purposes only and that various modifications or changes in light will be suggested to persons skilled in the art and are to be included within the spirit and purview of this application and the scope of the appended claims.

### EXAMPLES

### Example 1

### Vector-Based Generation of Nesiritide ORF

Primer-based codon addition was performed by PCR mutagenesis as follows—

| | | | Product | Digested | | |
|---|---|---|---|---|---|---|
| | | | size in | with | | |
| PCR | Template | Primers | bp | enzymes | Ligated to | Created |
| I | pRA | Seq ID 4 | 356 | Age I & | PCR I digested | pRA-N-I |
| | vector | Seq ID 5 | | Xho I | fragment ligated with | (Has 4 |
| | | | | | 3745 bp fragment of | A.As of |
| | | | | | pRA (digested with | Nesiritide) |
| | | | | | Age I & Xho I) | |
| II | pRA-N-I | Seq ID 4 | 364 | Age I & | PCR II digested | pRA-N-II |
| | | Seq ID 6 | | Xho I | fragment ligated with | (Has 8 |
| | | | | | 3745 bp fragment of | A.As of |
| | | | | | pRA (digested with | Nesiritide) |
| | | | | | Age I & Xho I) | |
| III | pRA-N- | Seq ID 4 | 374 | Age I & | PCR III digested | pRA-N-III |
| | II | Seq ID 7 | | Xho I | fragment ligated with | (Has 11 |
| | | | | | 3745 bp fragment of | A.As of |
| | | | | | pRA (digested with | Nesiritide) |
| | | | | | Age I & Xho I) | |
| IV | pRA-N- | Seq ID 4 | 389 | Age I & | PCR IV digested | pRA-N-IV |
| | III | Seq ID 8 | | Xho I | fragment ligated with | (Has 16 |
| | | | | | 3745 bp fragment of | A.As of |
| | | | | | pRA (digested with | Nesiritide) |
| | | | | | Age I & Xho I) | |
| V | pRA-N- | Seq ID 4 | 389 | Age I & | PCR V digested | pRA-N-V |
| | IV | Seq ID 9 | | Xho I | fragment ligated with | (Has 22 |
| | | | | | 3745 bp fragment of | A.As of |
| | | | | | pRA (digested with | Nesiritide) |
| | | | | | Age I & Xho I) | |
| VI | pRA-N-V | Seq ID | 425 | Age I & | PCR VI digested | pRA-N-VI |
| | | 10 | | Kpn I | fragment ligated with | (Has 26 |
| | | Seq ID 9 | | | 3725 bp fragment of | A.As of |
| | | | | | pRA (digested with | Nesiritide) |
| | | | | | Age I & Kpn I) | |
| VII | pRA-N- | Seq ID 4 | 435 | Age I & | PCR VII digested | pRA-N-VII |
| | VI | Seq ID | | EcoR I | fragment ligated with | (Has 30 |
| | | 11 | | | 3717 bp fragment of | A.As of |
| | | | | | pRA (digested with | Nesiritide) |
| | | | | | Age I & EcoR I) | |
| VIII | pRA-N- | Seq ID 4 | 449 | Age I & | PCR VIII digested | pRA-N- |
| | VII | Seq ID | | Hind III | fragment ligated with | VIII (Has |
| | | 12 | | | 3717 bp fragment of | 31 A.As of |
| | | | | | pRA (digested with | Nesiritide |
| | | | | | Age I & EcoR I) | & 2 stop |
| | | | | | | codons) |
| IX | pRA-N- | Seq ID | 413 | Age I & | PCR IX and PCR X | pRA-N |
| | VIII | 10 | | Stu I | digested fragments | (Has 32 |
| | | Seq ID | | | ligated with 3717 bp | A.As of |
| | | 13 | | | fragment of pRA | Nesiritide |
| X | pRA-N- | Seq ID | 975 | Stu I & | (digested with Age I | & 2 stop |
| | VIII | 14 | | Pvu I | & Pvu I) | codons) |
| | | Seq ID | | | | |
| | | 15 | | | | |

The primers were designed on a synthetically generated nesiritide ORF with convergent free energy of RNA based on a sequence that passed energy minimization of the transcript. Other criteria for further optimisation was the choice of leader sequences from E. Coli β-Galactosidase protein.

### Example 2

### Making of pRAZ-6-N

To make the chimeric Nesiritide construct, the following strategy was carried out—Using a construct created inhouse (which has the first 124 bases of the E. coli β-galactosidase gene as a fusion with a heterologous gene) as the template, carried out a PCR using primers SEQ ID No 16 (located at positions 287-337 on the template and has Sal I site) and SEQ ID No 17 (located at positions 441 to 472 on the template, has Sal I site}. Purified the 186 bp PCR product. Digested with Sal I in Buffer containing 50 mMTris-HCl, pH 7.5, 10 mM MgCl2, 100 mM NaCl and 0.1 mg/ml BSA at 37° C. overnight and purified the digested 145 bp FRAGMENT 1 using the Qiaquick PCR purification kit (Qiagen).

Digested pRA-N with Sal I in Buffer containing containing 50 mMTris-HCl, pH 7.5, 10 mM MgCl2, 100 mM NaCl and 0.1 mg/ml BSA at 37° C. overnight and purified the linear 4109 bp FRAGMENT 2 using the Qiaquick PCR purification kit (Qiagen). Ligated Fragment 1 with Fragment 2 with 2 units of T4 DNA Ligase in presence of buffer containing 40 mM Tris HCl, 10 mM MgCl2, 10 mM DTT and 0.5 mM ATP (pH 7.8) at 37° C. for 2 hrs and overnight at 4-12° C. to pRAZ-6-N vector (4254 bp).

### Example 3

### Making of pET-RAZ-6-N

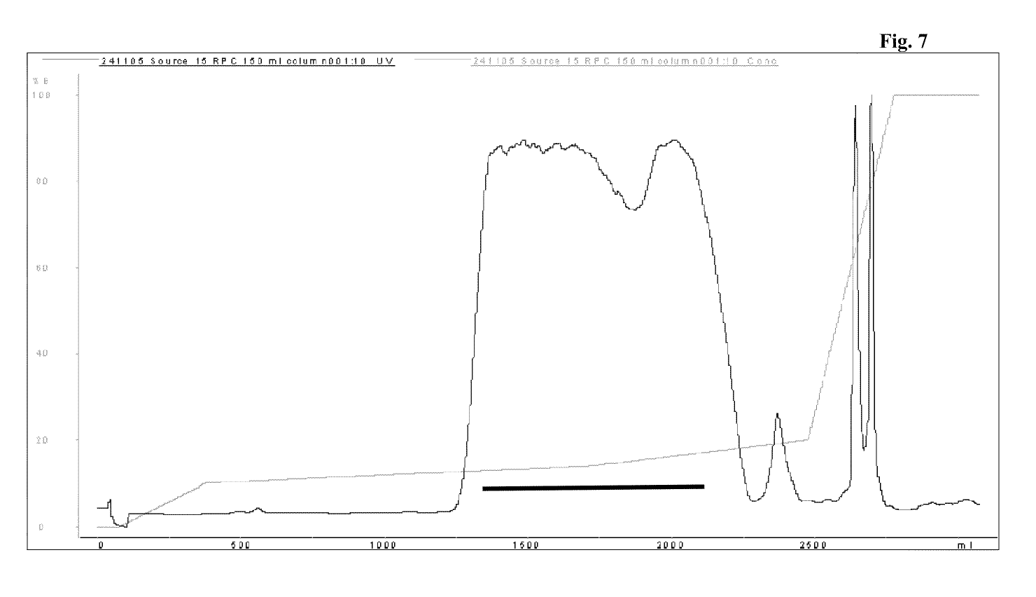

Using pRAZ-6-N as the template, carried out a PCR using primers Seq ID No 18 (located at positions 2-34 on the template) and Seq ID No 19, (located at positions 464 to 493 on the template and has Aat II site). Purified the 492 bp PCR product (FIG. 7) using Qiaquick PCR purification kit (Qiagen). Digested with NcoI and Aat II in buffer containing 33 mM Tris-acetate, pH7.9, 10 mM Mg-acetate, 66 mM K-acetate

and 0.1 mg/ml BSA at 37° C. overnight and purified the digested 161 bp FRAGMENT 3 and not the 331 bp fragment using Qiaquick PCR purification kit (Qiagen).

Using pRAZ-6-N as the template, carried out a PCR using primers Seq ID No 20 (located at positions 463-492 on the template and has Aat II site) and Seq ID No 21 (located at positions 634 to 679 on the template and has Xho I site). Purified the 217 bp PCR product using Qiaquick PCR purification kit (Qiagen). Digested with Aat II and Xho I in buffer containing 33 mM Tris-acetate, pH7.9, 10 mM Mg-acetate, 66 mM K-acetate and 0.1 mg/ml BSA at 37° C. overnight and purified the digested 174 bp FRAGMENT 4 using Qiaquick PCR purification kit (Qiagen).

Ligated FRAGMENT 3 with FRAGMENT 4 with 2 units of T4 DNA ligase in presence of buffer containing 40 mM Tris HCl, 10 mM MgCl₂, 10 mM DTT and 0.5 mM ATP (pH 7.8) at 37° C. for 2 hours and overnight at 4° C. and purified the 335 bp ligated FRAGMENT 5 using Qiaquick PCR purification kit (Qiagen). Digested pET 19b vector with Nco I and Xho I in buffer containing 10 mM Tris-HCl, pH 7.5, 10 mM MgCL2, 100 mM NaCl and 0.1 mg/ml BSA at 37° C. overnight and purified the 5643 bp FRAGMENT 6 and not the smaller 74 bp fragment using Qiaquick PCR purification kit (Qiagen).

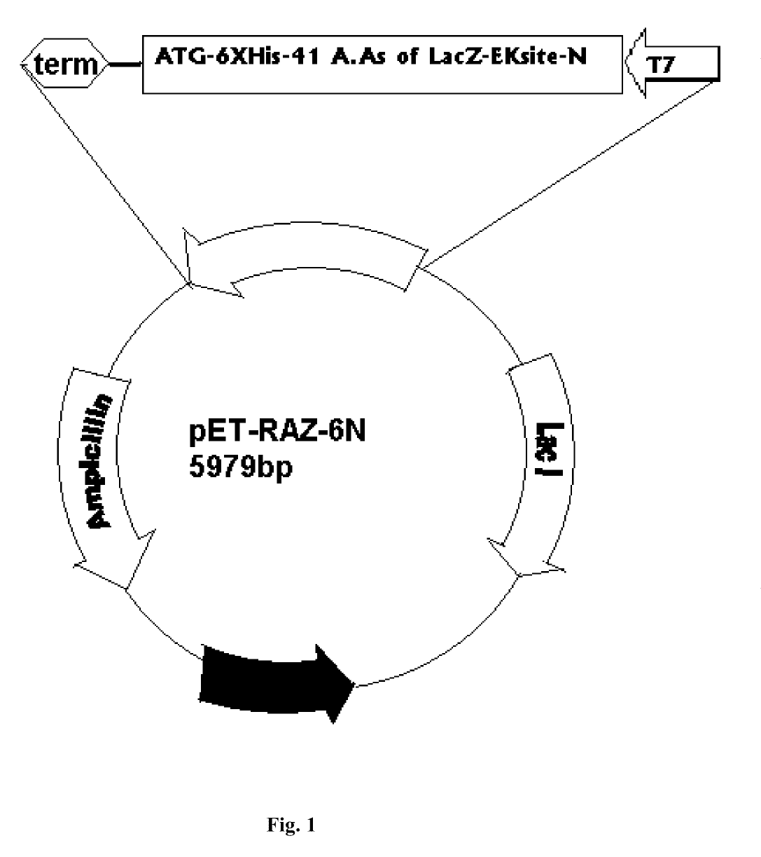

Ligated FRAGMENT 5 with FRAGMENT 6 with 2 units of T4 DNA ligase in presence of buffer containing 40 mM Tris HCl, 10 mM MgCl₂, 10 mM DTT and 0.5 mM ATP (pH 7.8) at 37° C. for 2 hours and overnight at 4° C. to get pET-RAZ-6-N vector (5979 bp) (Seq. ID NO:22 & FIG. 1).

This construct has the T7 promoter with 41 amino acids of the N-terminal region of β-Galactosidase protein linked to the Enterokinase protease cut site followed by the gene coding for Nesiritide, T7 transcription termination region, gene for ampicillin resistance, pBR322 origin and the LacI gene.

### Example 4

### Transformation of Bacterial Cells

The ligation mix from example 3 was used to transform competent bacterial hosts (DH5α, Top 10, LMG19, JM109, BL21 DE3 cells). The required amounts of competent cells were thawed on ice. The required amount of DNA was transferred into the tube containing the competent cells and mixed gently. Care was taken not to pipette or vortex. It was left on ice for 15-30 mins. The cells were subjected to heat shock at 42° C./2 mins and left on ice for 5 mins. One ml of appropriate medium (without antibiotic) was added and the cells were grown at 37° C. for 1 hr with shaking. Cells were pelleted at 3000 rpm/5 mins. The pellet was resuspended in 100 ul appropriate medium (without antibiotic) and spread plated on an agar plate of appropriate medium (with antibiotic). Incubated plates in a 37° C. incubator for 12-18 hrs. Colonies obtained were picked up and inoculated in 3 ml of media containing appropriate concentration of antibiotic. Grew the cells at 37° C. for 14-18 hrs with shaking and the overnight cultures were subjected to miniprep analysis by alkaline lysis method.

### Example 5

### Extraction of Plasmid DNA (Miniprep) Using Alkaline Lysis Method

Cultures grown overnight (˜1.5 ml) were transferred into sterile labeled 1.7 ml eppendorf tubes and centrifuged at 3000 rpm/5 min in Force 7 centrifuge to pellet the bacterial cells. The supernatant was removed by aspiration under vacuum (also from cap), leaving the pellet as dry as possible. The pellet was resuspended in 100 ul of ice-cold Solution I (sterile GTE) by vigorous vortexing for 10 secs and 200 ul of freshly prepared Solution II (lysis) was added to each tube. The tubes were mixed by inverting several times without vortexing till solution was clear. The tubes were stored on ice for 5 min. and later 150 ul of ice-cold Solution III (neutralization) was added. The tubes were mixed by inverting several times, stored on ice for 5 min and centrifuged at 14000 rpm for 10 min at 4° C. in Eppendorf. The supernatant was transferred to a fresh tube, taking care not to transfer any precipitate.

Equal vol. (˜450 ul) of phenol:chloroform:isoamyl alcohol (25:24:1) was added to the supernatant, vortexed to mix and centrifuged at 14000 rpm for 5 min. at room temp. The upper aqueous layer containing the DNA was carefully pipetted out to a fresh tube to which was added 1/10^{th} volume of 3M sodium acetate, pH 5.2, to the DNA solution to precipitate the DNA. Two vols. (˜1 ml) of ice-cold 95% ethanol was added to the tube, kept at −70° C. for 30 mins and centrifuged at 14000 rpm for 20 min. at 4° C. in Eppendorf centrifuge. The supernatant was discarded and the pellet was washed once with 500 ul of ice-cold 70% ethanol. The pellet was air-dried and resuspended in 30 ul of TE buffer pH 8.0+2 ul RNase A (1.0 mg/ml). Incubated at 37° C. for 1 hour. The miniprep DNAs were subjected to restriction enzyme digestions to confirm the vector construction. Positive clones were sequenced using the ABI Prism 310 Genetic Analyser

### Example 6a

### Fermentation of E. coli Containing pRAZ-6-N and pET-RAZ-6-N Vectors

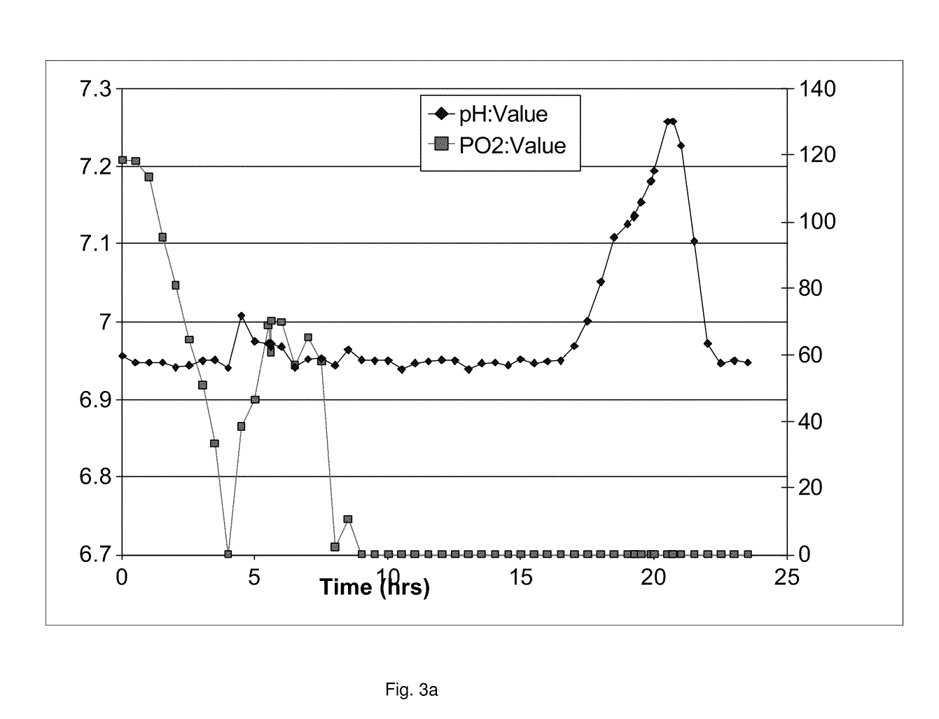

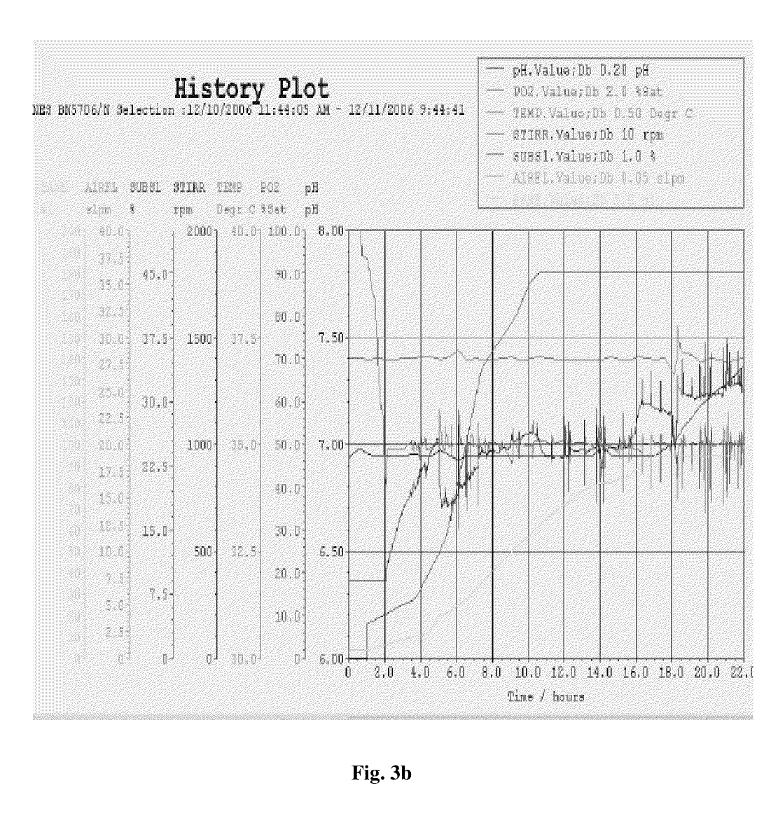

The culture from glycerol stock was streaked on a 2× Yeast Tryptone plate containing appropriate antibiotic which was incubated at 37° C. for 16-24 hrs. Single colony of the culture from the plate was inoculated in 10 ml of 2× Yeast Tryptone liquid medium and grown at 37° C., on rotary shaker (200 to 220 rpm) for 16 hrs. The grown culture was transferred to 100 mL of basal fermentation medium (seed medium) in a 500 mL conical flask and grown at 37° C., on a rotary shaker (200 to 220 rpm) for 8 hrs. 100 ml of seed culture was transferred to 900 ml of fermentation medium in 2 lt. jar fermentor procured from B Braun. The fermentation parameters were maintained as follows: aeration from 4 to 10 1 pm, temperature at 37° C., stirrer speed from 300 to 1200 rpm. pH was maintained between 6.9 to 8.0. pO₂ was cascaded with stirrer so as to maintain dissolved oxygen above 50% in the initial 16 hrs. The feed solution contained 15-25% glucose and 10-20% yeast extract. Fifteen to thirty ml of TES was added for 600 ml of feed. The feed was according to a predetermined feed rate which is a combination of exponential and linearly increasing feed rate. Nutrient solution was fed as per predefined strategy. Inducer solution was added between 12 to 20 hours of growth. Antifoam solution was fed as and when excess foaming was observed. At various time-points during the fermentation run (FIG. 3a), aliquots were withdrawn from the fermentor and the OD of the sample was determined by spectrophotometry. The final OD was ˜80 equivalent to ˜45 g dry cell weight. The chimeric protein production was about 15-20% of total cellular protein as determined by SDS-PAGE and was ˜1.7 g/L as estimated after initial capture stage. Without ORF optimisation, clones gave levels of protein which were not visualized on SDS-PAGE but were Western blot positive.

### Example 6b

A volume of 100 ul of Working Cell Bank culture was seeded in 20 ml of fermentor medium in 250 ml flask and grown to an O.D of 0.6 to 0.8 at 37° C., 200 rpm for 8 hrs. 20 ml of the seed created was aseptically added to another seed medium and grown to an O.D of 3-4 at 37° C. in 4 hrs. Fermentation media was prepared with dextrose, yeast extract and anti-foam and added to a C-15 fermentor. The media was sterilised at 121° C. for 25 mins. Base was added via a pump to adjust pH to 7.0. The DO was calibrated to 100%. Seed-2 was transferred to the fermentor operating under the following conditions: 30 1 pm air, 50% DO cascaded with a stirrer and 37° C. temperature. The feed was started at 1 hr and culture was induced at 15 hr with the inducer for 6-10 hrs (FIG. 3b).

### Example 7

### Homogenization of Cells

Approximately 1.2-1.6 liters of fermentation broth was harvested at the end of fermentation cycle. This was allowed to chill to about 8-10° C. on an ice bath. The cells were homogenised in the fermentation broth itself using a two-stage Niro Soavi high pressure homogeniser operated at 850-900 bars. The lysed material from each cycle was collected and the homogenisor washed with a 50 ml water. This was allowed to chill to 8-10° C. before start of next cycle. Three cycles of homogenisation was carried out to ensure maximal breakage of the cells. Alternatively, 15 L fermentor batch harvest was subjected to TFF by diluting in 25 litres of lysis buffer. The batch was concentrated to 7 litres and homogenised at 800 bar, 10° C. at the rate of 10 minutes per litre.

### Example 8

### Solubilization of Inclusion Bodies And Expanded Bed Chromatography

The volume of the cell lysate, obtained above was measured and solid urea crystal added (780 gms/L) to achieve 8M final concentration. The urea was allowed to dissolve using vigorous stirring and the solution was then gently stirred at ambient temperatures (22-25° C.) to allow complete solubilisation of the inclusion bodies containing the fusion protein. After 12-16 hours of gentle stirring, buffer components were added to give a final concentration of 100 mM sodium phosphate, pH 7.5; 300 mM sodium chloride and 80 mM of imidazole. This solution was diluted to twice the volume with water to bring urea concentration down to 4 M.

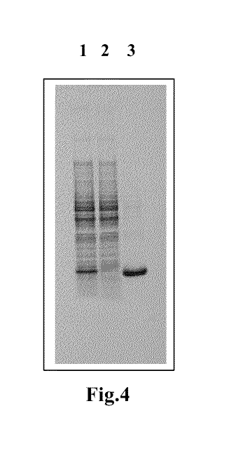

Streamline Chelating from Amersham was packed in a Streamline35 column and charged with Ni²⁺ in water. This column was equilibrated by pumping in buffer containing 50 mM sodium phosphate, pH 7.5; 150 mM sodium chloride; 40 mM of imidazole and 4 M urea and allowing the column bed to expand to 2-2.5 times the settled bed volume. The crude cell lysate was then pumped in at a certain flow rate to allow the bed height to remain below 3 times the settled bed height. After loading the entire cell lysate, the column was washed in expanded mode using 3 CV of equilibration buffer and the buffer flow stopped. The column was allowed to settle and further washed with 2 CV of equilibration buffer in settled bed mode till UV at 280 nm reached baseline. Bound protein was then eluted from the column in settled bed mode using the same buffer having increased Imidazole concentration (FIG. 4). Eluting Protein peak was monitored at 280 nm and collected in a single pool of approximately 2-2.5 CV.

### Example 9

### Digestion with Protease

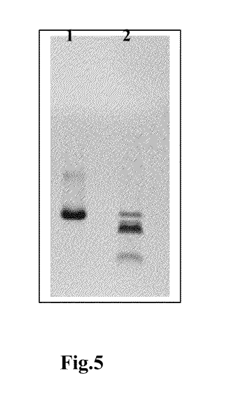

The protein pool from Example 8 was then desalted on a column of G-25 (Amersham) and equilibrated with 20 mM sodium phosphate, pH 7.5/4 M urea. The eluate from Streamline column was loaded and eluted with the same buffer. About 1.5 CV protein peak, monitored at 280 nm was collected and protein content measured using Bradford's Protein assay solution from Pierce. Fusion protein content of 1.2-1.6 gms was routinely obtained from 1 liter of culture broth. The protein concentration was adjusted to 5-10 mg/ml using the above buffer and the solution kept on ice bath under gentle stirring. After the temperature reaches 5-6° C., recombinant Enterokinase was added at a concentration of 20,000 units per gram of fusion protein. Release of b-type natriuretic peptide was carried out under gentle stirring at 5-6° C. for 16 hours (FIG. 5). The extent of b-type natriuretic peptide released is quantitated by comparing the peak area obtained by injecting a small aliquot of the digestion mixture onto a C18 reverse phase column and that obtained from a known amount of standard peptide. The solution was removed from the cold and allowed to attain ambient temperature (22-25° C.) and then microfiltered using 0.45 micron hollow fiber filter.

### Example 10

### Ion Exchange Chromatography

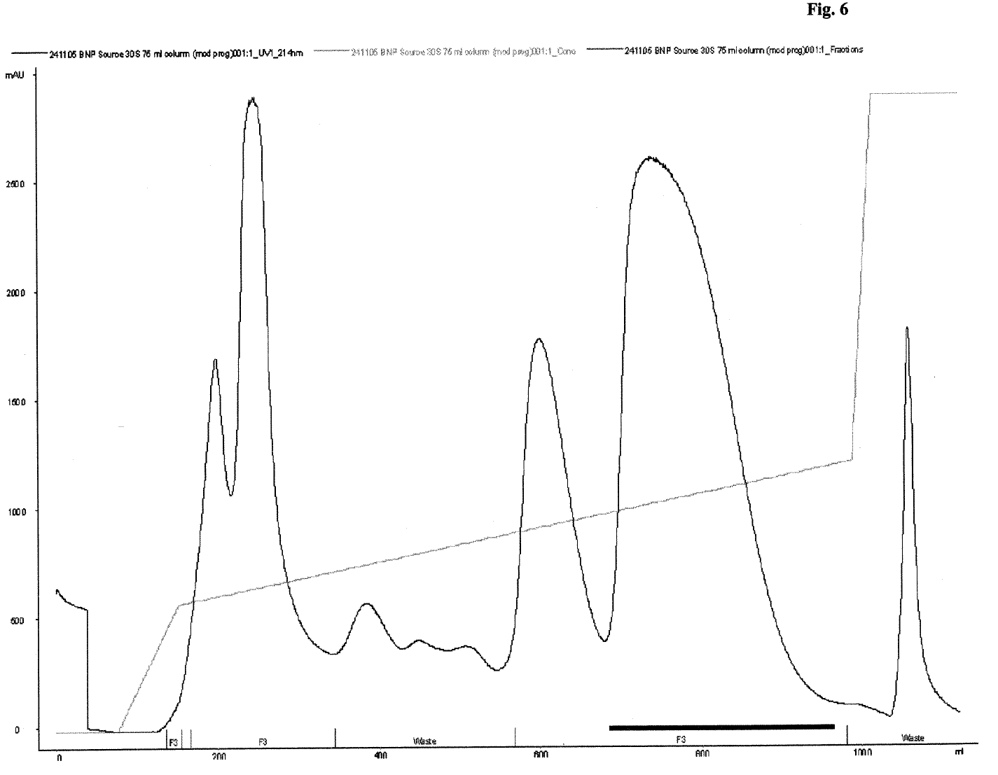

The solution obtained from Example 9 was loaded onto a column of Source 30S (Sulfopropyl ion exchanger; Amersham). The column was previously equilibrated with 20 mM sodium phosphate, pH 7.5/4 M Urea. The column was washed first with 2 CV of equilibration buffer followed by 2 CV of 20 mM sodium phosphate, pH 7.5 and finally with 2 CV of 20 mM sodium phosphate, pH 7.5 containing 5-30% Acetonitrile (buffer A). Bound proteins were eluted with a gradient between buffer A and Buffer B (20 mM sodium phosphate, pH 7.5/10-80% Acetonitrile/200-500 mM NaCl) using different gradient programs. Elution of peptide from the column was monitored at 214 nm and the peak eluting between 17-20 mS (FIG. 6) was pooled for further purification. Peptide content was determined by RP-HPLC with >95% purity.

### Example 11a

### Hydrophobic Interaction Chromatography

The eluate obtained from the cation exchange chromatography column was fed to a 100 mm diameter column packed with 1 litre of Source Phe matrix. The column was equilibrated with 3 litres of Equilibration buffer (Buffer A) which consisted of 50 mM sodium phosphate (pH 7.4) and 2M ammonium sulphate at a flow rate of 200 ml/min. The eluate loaded on the column was then washed with 2 litres of Buffer A at the same flow rate. Protein was eluted using a linear gradient of 0-20% Buffer B (water) for 25 mins and 20-40% Buffer B (125 min at a flow rate of 80 ml/min). Peptide peak was monitored at 214 nm. The peak obtained was fractionated and fractions having purity greater than 99% were pooled. The pooled fractions were

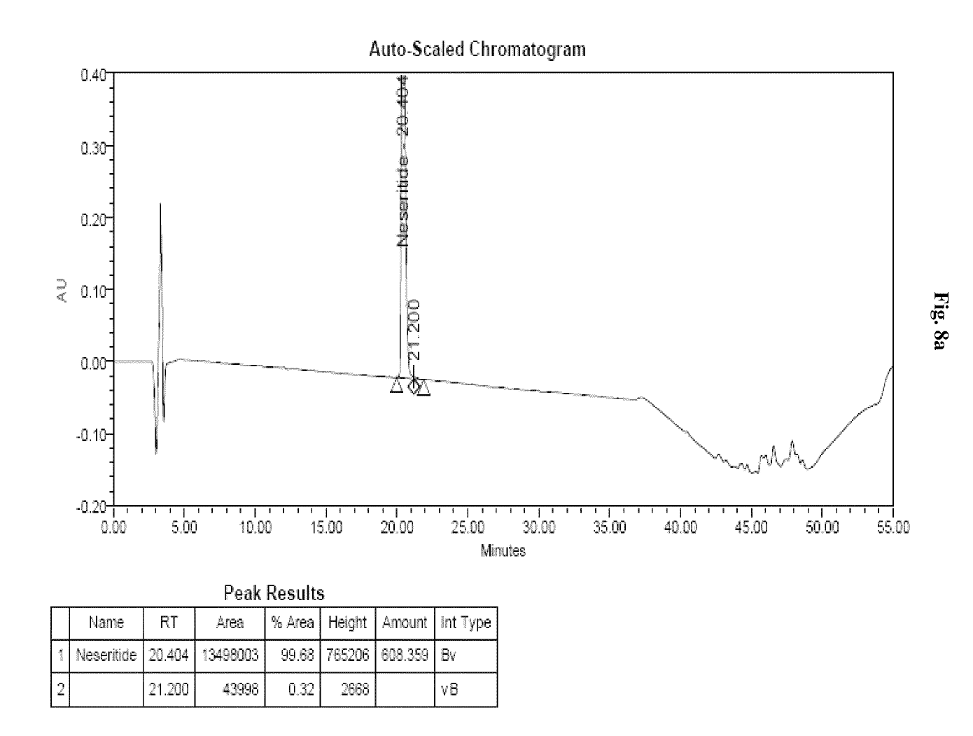

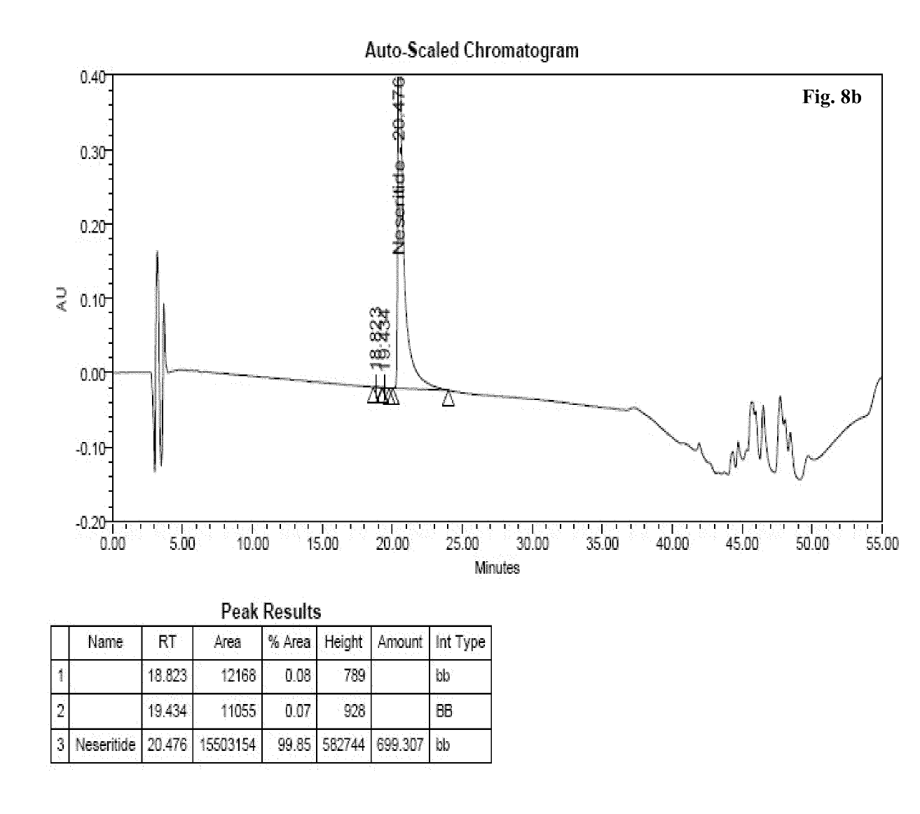

diluted 1:1 with 0.05 M sodium phosphate and 3M ammonium sulphate and loaded onto a 300 ml column of Source Phenyl matrix equilibrated with 0.05 M sodium phosphate buffer, pH 8.0 and 2M ammonium sulphate. The column was washed with 2 Column volumes of equilibration buffer and the bound peptide was eluted with 10 mM Sodium acetate, pH 5.0. The peptide peak was monitored at 214 nm. Peptide content and purity was determined by RP-HPLC (FIGS. 8a & 8b).

### Example 11b

### Reverse Phase Chromatography

The peptide peak obtained in Example 10 was diluted 1:1 with USP grade water and loaded onto a column of Source 15RPC (Amersham) equilibrated with 10 mM sodium phosphate buffer, pH adjusted to 2.0 with phosphoric acid (buffer A). The column was washed with the same buffer and bound peptide eluted with a gradient of buffer A to B (60-80% acetonitrile in water). The peptide peak eluting between 13-17% was pooled (FIG. 7) and analysed by RP-HPLC and determined to be >99%

pure. The peptide peak obtained above was diluted 1:1 with water and loaded onto a column of Source 30RPC. After sample loading was complete, the column was washed with 10 CV of 0.1% acetic acid in water and the bound peptide eluted with a linear 5 CV gradient of 60-80% Acetonitrile in water. The peptide peak was pooled and lyophillised. Table 4 shows purification profile of Nesiritide from 1 L culture.

TABLE 4
Purification of Nesiritide from 1 L culture

| Step | Protein (mg) | % Purity |
|---|---|---|
| Fusion protein | 1386 | — |
| Released BNP after EK | 113 | 19.36 |
| digestion | | |
| BNP after Source 30S IEX step | 81 | 95.92 |
| BNP after Source 15RPC step | 61 | 99.14 |
| Concentration and | 56 | 99.44 |
| lyophilisation | | |

### Example 12

### Protein Precipitation

As an alternative to ion-exchange chromatography for purification, the more soluble peptide could be easily isolated from the fusion tag and the uncleaved fusion protein and other impurities by a simple pH precipitation method. Fusion protein was purified, desalted and enzymatically cleaved as described in example 9. The cleavage reaction solution was allowed to attain ambient temperature (22-25° C.) and then diluted 5 folds with 20 mM sodium phosphate, pH 7.5. Dilute (1-2 N) phosphoric acid was added slowly under stirring to bring down the pH to 5.0-5.5. The copious protein precipitate formed could be easily pelleted by centrifugation at 5000×g for 20 min and the clear supernatant that contained >95% of the peptide was collected. Alternatively, the soluble peptide could also be obtained by a novel technique of microfiltration through 0.22 u tangential flow filter (Pall) of the pH adjusted solution. The inlet pressure was maintained at <1 bar with a recirculation rate of 180-200 ml/min. The clear filtrate was collected and the turbid retente washed with 100 ml of 20 mM phosphate buffer, pH 5.5. The washings were pooled with the original filtrate and peptide content estimated by RP-HPLC. Recovery of the peptide was calculated to be >95% at this step. Further purification of the peptide was carried by reverse-phase chromatograpy.

### Example 13

### (a) Analytical RP-HPLC

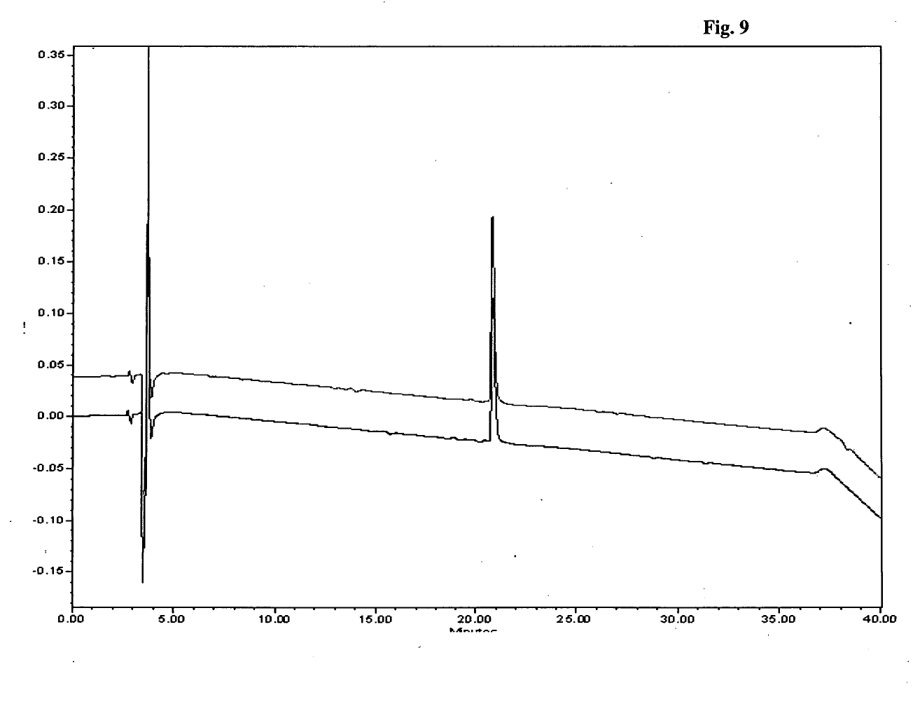

Enterokinase cleavage to release b-type natriuretic peptide and its subsequent purification was analysed by the following analytical method—Reverse phase chromatography on Inertsil WP C18 (4.6×250 mm; 5 micron; 300 A) column with the mobile phase system which was as follows: A=0.1% v/v TFA in water, B=0.1% TFA v/v in 75% Acetonitrile. Column oven temperature was maintained at 30° C. and sample chamber at 5° C. Flow rate of 1 ml/min was used throughout the run. Absorbance was monitored at 210 nm (FIGS. 9a & 9b). Synthetic b-type natriuretic peptide (1-32) from Bachem was used as a reference standard in all HPLC assays.

### (b) Tryptic Mapping

Tryptic digestion was carried out using 0.5 ml of purified peptide at 2 mg/ml concentration dissolved in 50 mM Tris.Cl, pH 7.5 buffer. TPCK treated trypsin (sequencing grade; Roche) was used for digestion at 1:67 w/w (0.015 ml of 1 mg/ml per mg of peptide) and incubated at 37° C. for 4 hrs. Standard BNP was also treated with trypsin as mentioned above. The digestion mixture was spun at 12,000 rpm for 15 min and 20 ul aliquots of each, equivalent to 40 ug peptide was injected onto Inertsil WP C18 column and eluted using the following gradient program (Table 5):

TABLE 5
Gradient programme for RP-HPLC

| Time (min) | % A | % B |
|---|---|---|
| 0 | 0 | 0 |
| 5 | 100 | 0 |
| 10 | 85 | 15 |
| 53 | 55 | 45 |
| 60 | 20 | 80 |
| 65 | 20 | 80 |
| 70 | 0 | 100 |
| 75 | 0 | 0 |

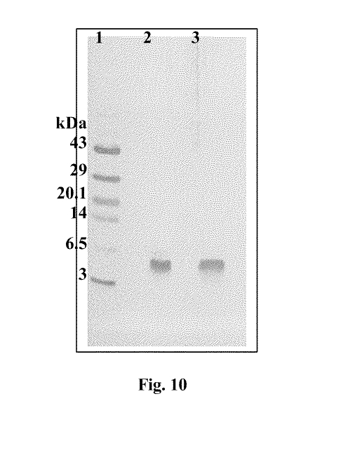

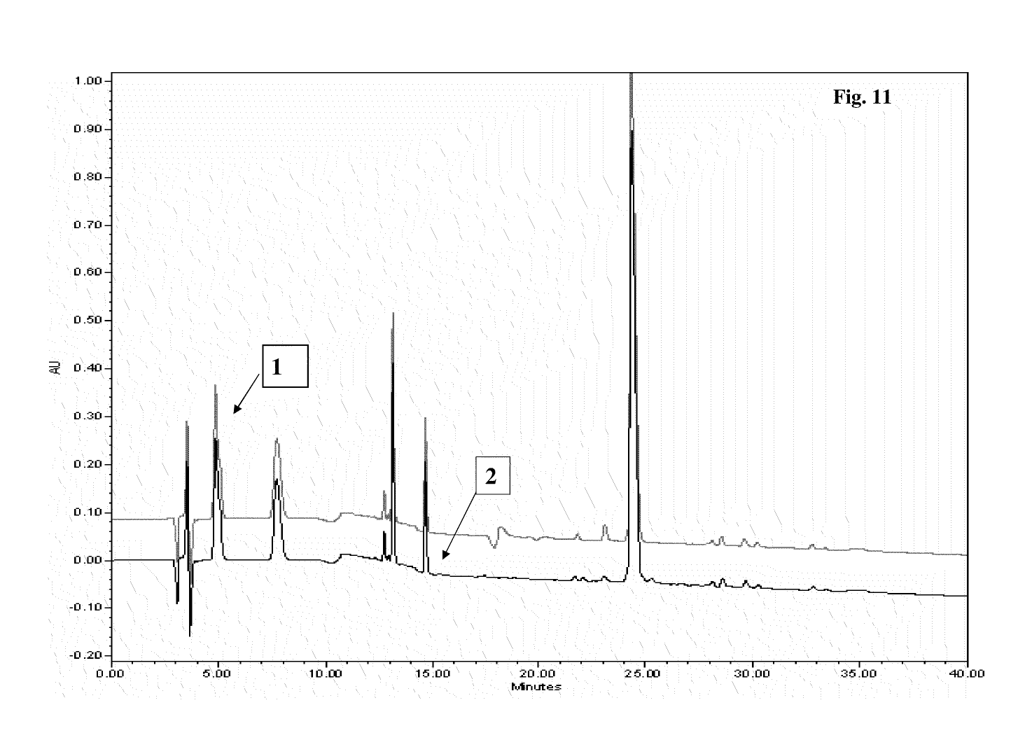

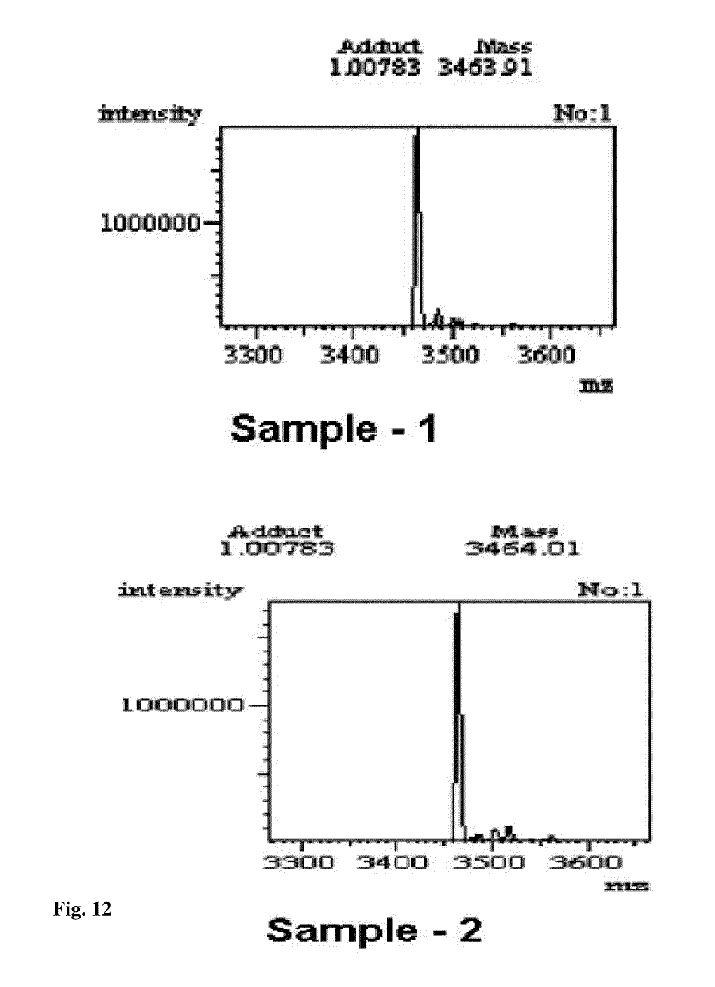

All HPLC data was collected on a Waters system with PDA detector. The purified peptide (FIG. 10) was also analysed by N-terminal sequencing, amino acid analysis, Tryptic digestion (FIG. 11) and ESI-Mass (FIG. 12) and found to completely match data for the reference standard BNP.

### Example 14

### In-vitro Bioassay of Recombinant Nesiritide

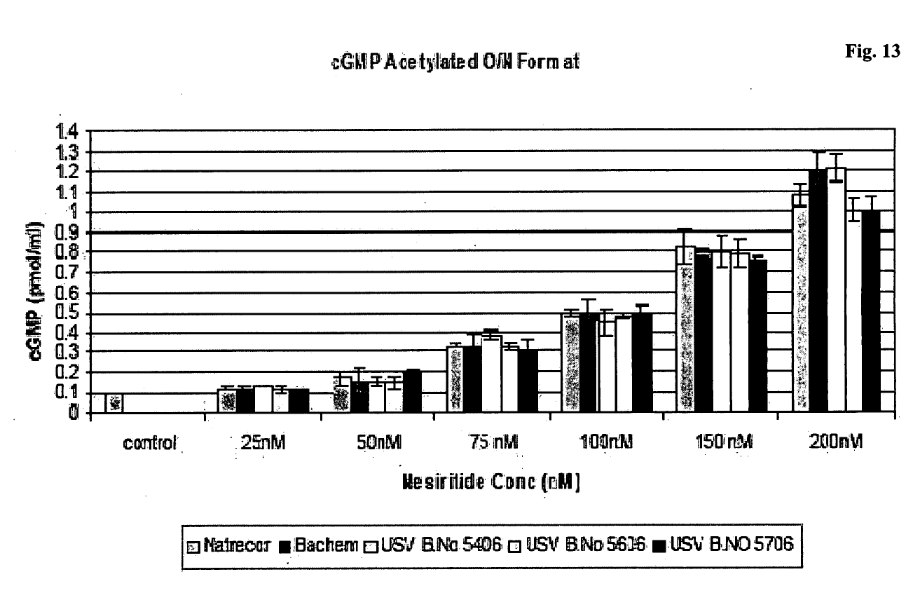

Nesiritide is known to elicit its biological activity via a cGMP second messenger system in vitro and in vivo. PC-12 cells were plated in Plain RPMI medium in 24-well plate and equilibrated at 37° C., 5% CO₂ incubator for 2 hrs. Prestimulation medium (RPMI+0.1% BSA+0.1 mM IBMX) was added to cells for 10 mins. It was followed by the addition of different concentrations of Nesiritide for 30 mins at 37° C., 5% CO₂ incubator. The cells were lysed with 0.1M HCL for 20 mins followed by 5-7 freeze-thaw cycles. The cell lysates were centrifuged in microfuge at 10000 rpm for 10 mins at 4° C. Protein concentration of the supernatant was estimated by Bradford's method. The supernatant from cell lysates was assayed for cGMP concentration (FIG. 13) using a cGMP kit (Assay Design Inc). The cGMP from cell lysates was estimated from cGMP standard curve. ED50 value was calculated. Table 6 shows a comparative analytical data for ED50 values performed for Natrecor (Innovator formulation) used as reference standard, synthetic nesiritide (Bachem) and 3 batches of recombinant nesiritide of the present invention.

TABLE 6
Effect of Nesiritide samples on cGMP levels in PC-12 cells

| | | cGMP levels (pmol/ml) (+/− | | | | |
|---|---|---|---|---|---|---|
| | Nesiritide | | | USV | USV | USV |
| | Conc | | | B. No | B. No | B. No |
| Sr. No | (nM) | Natrecor | Bachem | 5406 | 5606 | 5706 |
| 1 | Control | 0.095 +/− 0.004 | 0.095 +/− 0.004 | 0.095 +/− 0.004 | 0.095 +/− 0.004 | 0.095 +/− 0.004 |
| 2 | 25 nM | 0.126 +/− 0.01 | 0.121 +/− 0.006 | 0.133 +/− 0.004 | 0.119 +/− 0.01 | 0.114 +/− 0.005 |
| 3 | 50 nM | 0.168 +/− 0.02 | 0.164 +/− 0.05 | 0.161 +/− 0.01 | 0.150 +/− 0.03 | 0.191 +/− 0.01 |
| 4 | 75 nM | 0.327 +/− 0.009 | 0.327 +/− 0.05 | 0.383 +/− 0.02 | 0.321 | 0.305 +/− 0.05 |
| | | | | | 0.02 | |
| 5 | 100 nM | 0.499 +/− 0.02 | 0.502 +/− 0.05 | 0.444 +/− 0.06 | 0.475 +/− 0.01 | 0.478 +/− 0.06 |
| 6 | 150 nM | 0.823 +/− 0.08 | 0.793 +/− 0.02 | 0.799 +/− 0.08 | 0.787 +/− 0.07 | 0.756 +/− 0.02 |
| 7 | 200 nM | 1.08 +/− 0.05 | 1.206 +/− 0.08 | 1.214 +/− 0.06 | 1.006 +/− 0.05 | 1.085 +/− 0.06 |
| | *ED₅₀ | 75.78 | 74.27 | 77.45 | 79.0 | 79.09 |
| | (nM) | | | | | |

*Acceptable range: ED 50 value 70-85 nM

### Example 15

### Nesiritide ELISA

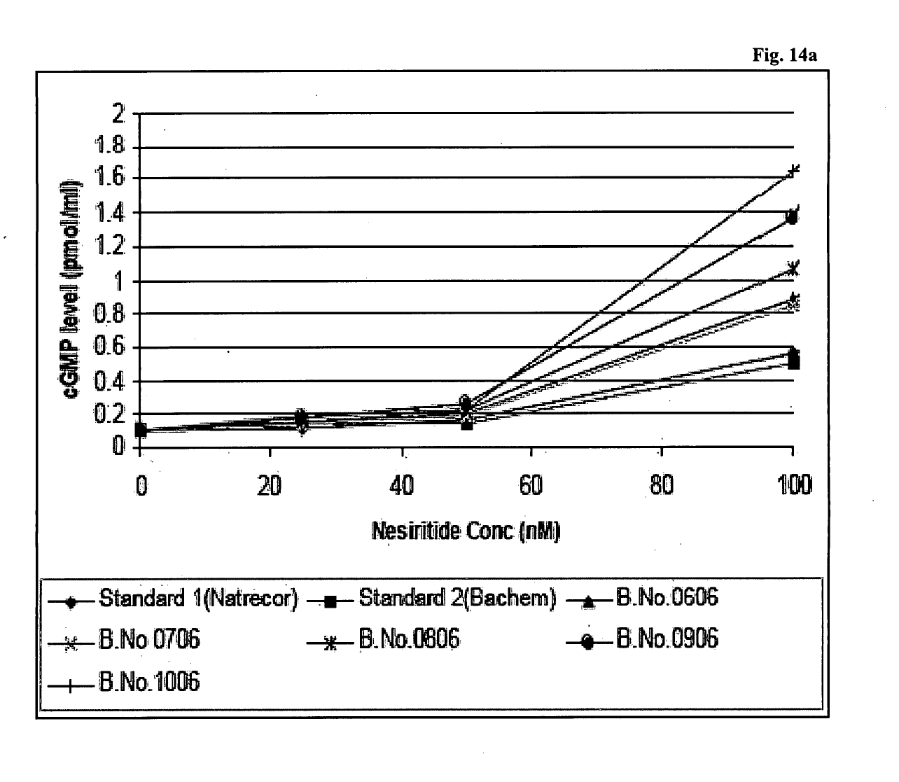

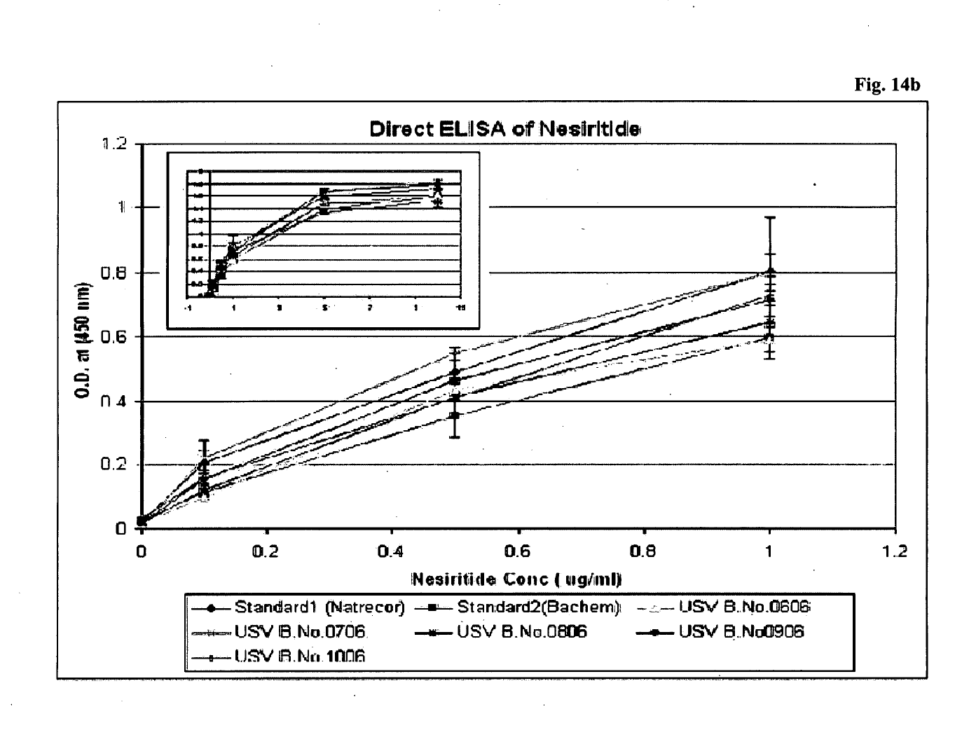

Different concentrations of recombinant human Nesiritide (0.1 ug/ml to 10 ug/ml) diluted in Carbonate/Bicarbonate buffer pH 9.6 were incubated in 96-well plate overnight at room temperature. Following the incubation, wells were washed with PBS and blocked with 1% BSA for 4-6 hours. It was followed with PBS wash and the wells were incubated overnight at 4° C. with rabbit anti-human Nesiritide antibody (1:1000) diluted in 0.1% BSA in PBS. The wells were then washed four times with PBS containing Tween-20 followed by incubation with anti rabbit HRPO labeled antibody for 90 minutes at RT. The wells were washed four times with PBS+Tween-20 followed by addition of TMB substrate and incubated for 20-30 minutes. The reaction (development of blue color) was stopped with 2M H₂SO₄ and the plate was read at 450 nm. The optical values were plotted against the concentration of Nesiritide (FIGS. 14a & 14b).

As seen in the above examples, the chimeric construct of the field of this invention on expression and purification from E. coli cells at 1.0-8 g/litre gave an acetate peptide of Nesiritide suitable for therapeutic use.

We intend the legal coverage of our patent to be defined not by the Examples of the Specification, but by our patent claims; we provide Examples to enable scientists to practice our invention, not to enable lawyers to define the legal coverage of our patent.

Routine variations of our claimed invention will be readily apparent to one of skill in the art. For example, claim 83 covers a process wherein chimera polypeptide is bound to, then separated from, an affinity binder. The affinity binder may be a chromatography resin, a latex-bead linked antibody anti-chimera monoclonal antibody, a solid-phase Fmoc synthesis resin, et cetera. The affinity binder can bind the chimera reversibly (e.g., with an ionic bond). Alternatively, the affinity binder can bind the chimera polypeptide irreversibly (e.g., where the affinity binder is a certain type of solid phase Fmoc resin), so the chimera polypeptide is unbound by cleaving it, for example by including in the chimera polypeptide a dedicated affinity binder-binding sequence from which the chimera polypeptide is cleaved.

The claim term “comprising” is used in its accepted meaning as allowing, but not requiring, the addition of additional ingredients or components. For example, the phrase “comprising . . . a sequence” encompasses that sequence alone or combined with other residues.

A “homologous” sequence is a sequence which appears in the wild-type organism. A “heterologous” sequence is a sequence which does not appear naturally in an organism in which the homologous sequence exists.

Increasing a polypeptide expression product which is recoverable from an expression system may be caused by increasing the rate of transcription or translation, increasing the efficiency of transcription, or decreasing the rate of degradation of an expression product (e.g., mRNA or polypeptide). The precise mechanism does not limit the appended claims.

We claim:

1. An improved process for synthesis of Nesiritide comprising:
a. preparing a synthetic cDNA construct as set forth in Seq. ID No. 1 encoding ORF of Nesiritide polynucleotide by iterative optimization of RNA transcript free energy operably in the range of (−)30 Kilo calories to (−)300 Kilo calories per mole;
b. fusing said polynucleotide of step a) with a fusion partner consisting essentially of 41 amino acids from the N-Terminal region of E. coli beta-galactosidase and an affinity handle operably linked to regulatory elements cloned in an expression vector;
c. expressing the synthetic cDNA chimera as set forth in Seq. ID No. 3 in a host by culturing the host cells in the growth medium under appropriate conditions to yield Nesiritide chimeric protein; and
d. isolating and purifying the Nesiritide obtained from step c.

2. The process as claimed in claim 1, wherein said synthetic cDNA construct as set forth in Seq. ID No: 1 is prepared by sequential PCR cloning technique.

3. The process as claimed in claim 2, wherein the sequential PCR cloning technique is primer driven codon optimization of the gene.

4. The process as claimed in claim 3, wherein the gene-specific primers are selected from Seq. ID No:4 to Seq. ID No:21.

5. The process as claimed in claim 1, wherein the expression vector consists of synthetic cDNA chimeric sequence as set forth in Seq. ID No:3.

6. The process as claimed in claim 5, wherein the expression vector is pET-RAZ-6-N having sequence as set forth in Seq. ID No:22 with cDNA chimeric sequence from 5315 bp to 5607 bp.

7. The process as claimed in claim 1, wherein the affinity handle is selected from maltose-binding protein, poly Histidine and staphylococcal protein.

8. The process as claimed in claim 7, wherein the affinity handle is poly histidine.

9. The process as claimed in claim 1, wherein the host is E. coli

10. The process as claimed in claim 9, wherein E. coli harbours the expression vector pET-RAZ-6-N as set forth in Seq. ID No. 22 having cDNA chimeric sequence from 5315 bp to 5607 bp, said vector comprising synthetic cDNA chimeric sequence as set forth in Seq. ID No:3.

11. The process as claimed in claim 1, wherein the expression of synthetic cDNA chimera as set forth in Seq. ID No. 3 which encodes Nesiritide is under the control of a promoter, selected from a group consisting of araBAD, trp, T7, lac, pho, and trc.

12. The process as claimed in claim 11, wherein the promoter is T7.

13. The process as claimed in claim 1, wherein said growth medium comprises an inducer selected from a group consisting of Isopropyl-beta-D-thiogalactopyranoside (IPTG), lactose, maltose, arabinose and arabino galactan.

14. The process as claimed in claim 13, wherein the inducer is lactose.

15. The nesiritide synthetic cDNA chimera as set forth in Seq. ID No:3 prepared by the process as claimed in claim 1.

16. The process as claimed in claim 1, wherein the yield of nesiritide chimeric protein is in the range of 1 g/L to 8 g/L.

17. The process as claimed in claim 1, wherein the isolation and purification is carried out by sequential steps comprising:
a. isolating and solubilizing the inclusion bodies;
b. capturing the Nesiritide chimeric protein from the solubilized inclusion bodies;
c. digesting said protein of step b by proteases;
d. precipitating the impurities to separate out Nesiritide in solution; and
e. purifying the Nesiritide from the solution of step d by chromatography.

18. The process as claimed in claim 17, wherein the nesiritide chimeric protein is captured by expanded bed chromatography.

19. The process as claimed in claim 17, wherein proteolytic digestion is carried out by proteases selected from the group consisting of factor Xa, enterokinase, thrombin and trypsin.

20. The process as claimed in claim 19, wherein the protease is enterokinase.

21. The process as claimed in claim 17, wherein impurities are precipitated out by adjusting pH to about 5.0 to 5.5.

22. The process as claimed in claim 17, wherein nesiritide from the solution is purified by RP-HPLC or HIC, followed by desalting and buffer exchange.

23. The process as claimed in claim 22, wherein the purified Nesiritide is having mass of 3464 daltons and purity of at least about 99%.

24. The process as claimed in claim 22, wherein the purified Nesiritide is having ED50 in the range of 70 nM to 85 nM.

25. A process for large scale purification of nesiritide comprising:
a. solubilizing the inclusion bodies by treating with 8M Urea;
b. capturing the nesiritide chimeric protein using Iminodiacetic Acid (IDA) expanded bed chromatography;
c. digesting said protein of step b by enterokinase;
d. precipitating the impurities at a pH of about 5.0 to 5.5 to separate out Nesiritide in solution;
e. purifying the nesiritide from solution of step d by HIC;
f. desalting the pure nesiritide of step e by ion exchange chromatography; and
g. performing buffer exchange on desalted pure Nesiritide of step f

26. The process as claimed in claim 25, wherein the purity of Nesiritide is at least about 99.5%.

27. A sequential PCR cloning primer consisting of a nucleic acid sequence selected from the group consisting of: SEQ. ID No:4; SEQ. ID No:5; SEQ. ID No:6; SEQ. ID No:7; SEQ. ID No:8; SEQ. ID No:9; SEQ. ID No:10; SEQ. ID No:11; SEQ. ID No:12; SEQ. ID No:13; SEQ. ID No:14; SEQ. ID No:15; SEQ. ID No:16; SEQ. ID No:17; SEQ. ID No:18; SEQ. ID No:19; SEQ. ID No:20; and SEQ. ID No:21.

28. The PCR cloning primer of claim 27, comprising a primer having a sequence consisting of the sequence of SEQ. ID No:4.

29. The PCR cloning primer of claim 27, comprising a primer having a sequence consisting of the sequence of SEQ. ID No:5.

30. The PCR cloning primer of claim 27, comprising a primer having a sequence consisting of the sequence of SEQ. ID No:6.

31. The PCR cloning primer of claim 27, comprising a primer having a sequence consisting of the sequence of SEQ. ID No:7.

32. The PCR cloning primer of claim 27, comprising a primer having a sequence consisting of the sequence of SEQ. ID No:8.

33. The PCR cloning primer of claim 27, comprising a primer having a sequence consisting of the sequence of SEQ. ID No:9.

34. The PCR cloning primer of claim 27, comprising a primer having a sequence consisting of the sequence of SEQ. ID No:10.

35. The PCR cloning primer of claim 27, comprising a primer having a sequence consisting of the sequence of SEQ. ID No:11.

36. The PCR cloning primer of claim 27, comprising a primer having a sequence consisting of the sequence of SEQ. ID No:12.

37. The PCR cloning primer of claim 27, comprising a primer having a sequence consisting of the sequence of SEQ. ID No:13.

38. The PCR cloning primer of claim 27, comprising a primer having a sequence consisting of the sequence of SEQ. ID No:14.

39. The PCR cloning primer of claim 27, comprising a primer having a sequence consisting of the sequence of SEQ. ID No:15.

40. The PCR cloning primer of claim 27, comprising a primer having a sequence consisting of the sequence of SEQ. ID No:16.

41. The PCR cloning primer of claim 27, comprising a primer having a sequence consisting of the sequence of SEQ. ID No:17.

42. The PCR cloning primer of claim 27, comprising a primer having a sequence consisting of the sequence of SEQ. ID No:18.

43. The PCR cloning primer of claim 27, comprising a primer having a sequence consisting of the sequence of SEQ. ID No:19.

44. The PCR cloning primer of claim 27, comprising a primer having a sequence consisting of the sequence of SEQ. ID No:20.

45. The PCR cloning primer of claim 27, comprising a primer having a sequence consisting of the sequence of SEQ. ID No:21.

46. A DNA sequence comprising a sequence coding for a polypeptide of the sequence SEQ. ID. NO. 2, said DNA sequence having at least about fifteen percent engineered DNA residues, said engineered DNA residues differing from the DNA residues for wild type sequence for nesiritide.

47. A DNA sequence comprising residue about 1 to residue about 93 of SEQ ID NO. 1.

48. The DNA sequence of claim 46, wherein said DNA transcribes to an RNA with a transcriptional free energy of greater than about −200 Kcal/mol.

49. The DNA sequence of claim 46, wherein said DNA transcribes to an RNA with a transcriptional free energy of greater than about −100 Kcal/mol.

50. The DNA sequence of claim 46, wherein said DNA transcribes to an RNA with a transcriptional free energy of about −30 Kcal/mol.

51. A process comprising: expressing the DNA sequence of claim 46 to make a polypeptide comprising the polypeptide of SEQ. ID. NO. 2.

52. A process comprising: expressing the DNA sequence of claim 47 to make a polypeptide comprising the polypeptide of SEQ. ID. NO. 2.

53. A recombinant DNA sequence consisting essentially of residue about 1 to residue about 189 of SEQ ID NO. 3.

54. The DNA sequence of claim 53 located in the same reading frame as a DNA sequence coding for a heterologous polypeptide.

55. In a system for expressing a DNA sequence to produce a polypeptide of interest, the improvement comprising a DNA sequence coding for a pI modifier polypeptide, wherein:
a. said polypeptide of interest has a pI_{API}, said pI modifier polypeptide has a pI_{MOD}, and said pI_{MOD} differs from said pI_{API} so that at a precipitation pH, the polypeptide of interest can be separated from the pI modifier polypeptide by precipitation; and
b. the presence of the DNA sequence coding for the pI modifier polypeptide is effective to increase the amount of the polypeptide of interest which is recoverable from the expression system, when compared to the amount of polypeptide of interest which is recoverable in the absence of the DNA sequence coding for the pI modifier polypeptide.

56. The system of claim 55, said pI modifier polypeptide being about 123 amino acids in size.

57. The system of claim 56, said pI modifier polypeptide being at least a portion of beta-galactosidase polypeptide.

58. The system of claim 56, said DNA sequence coding for the pI modifier polypeptide comprising residue about 1 to residue about 189 of SEQ ID NO. 3.

59. The system of claim 55, said polypeptide of interest comprising the polypeptide of SEQ ID NO. 2.

60. The system of claim 55, wherein the DNA sequence coding for the polypeptide of interest is located in the same reading frame as the DNA sequence coding for the pI modifier polypeptide, so that the polypeptide of interest is expressed as a concatenated chimera polypeptide including the pI modifier polypeptide.

61. The system of claim 60, said DNA sequence coding for the pI modifier polypeptide being a DNA sequence coding for at least a portion of beta-galactosidase polypeptide.

62. The system of claim 60, said DNA sequence coding for a polypeptide comprising the polypeptide of SEQ ID NO. 2.

63. A method for increasing the expression of a DNA sequence coding for a polypeptide of interest comprising expressing said DNA sequence in the system of claim 55.

64. A method for increasing the expression of a DNA sequence coding for a polypeptide of interest comprising expressing said DNA sequence in the system of claim 57.

65. A method for increasing the expression of a DNA sequence coding for a polypeptide of interest comprising expressing said DNA sequence in the system of claim 59.

66. A method for increasing the expression of a DNA sequence coding for a polypeptide of interest comprising expressing said DNA sequence in the system of claim 60.

67. A method for increasing the expression of a DNA sequence coding for a polypeptide of interest comprising expressing said DNA sequence in the system of claim 62.

68. The DNA sequence of claim 46, further comprising residue about 1 to residue about 189 of SEQ ID NO. 3.

69. A polypeptide expression product of the DNA sequence of claim 68.

70. The DNA sequence of claim 68, wherein said DNA transcribes to an RNA with a transcriptional free energy of greater than about −200 Kcal/mol.

71. The DNA sequence of claim 68, wherein said DNA transcribes to an RNA with a transcriptional free energy of greater than about −100 Kcal/mol.

72. The DNA sequence of claim 68, wherein said DNA transcribes to an RNA with a transcriptional free energy of about −30 Kcal/mol.

73. The DNA sequence of claim 47, further comprising residue about 1 to residue about 189 of SEQ ID NO. 3.

74. A polypeptide expression product of the DNA sequence of claim 73.

75. The DNA sequence of claim 73, wherein said DNA transcribes to an RNA with a transcriptional free energy of greater than about −200 Kcal/mol.

76. The DNA sequence of claim 73, wherein said DNA transcribes to an RNA with a transcriptional free energy of greater than about −100 Kcal/mol.

77. The DNA sequence of claim 73, wherein said DNA transcribes to an RNA with a transcriptional free energy of about −30 Kcal/mol.

78. A process comprising expressing the DNA sequence of claim 68.

79. A process comprising expressing the DNA sequence of claim 73.

80. The system of claim 55, wherein the DNA sequence coding for the polypeptide of interest and the DNA sequence coding for the pI modifier polypeptide are in the same reading frame, so that the system expresses a chimera polypeptide comprising the polypeptide of interest and the pI modifier polypeptide.

81. The system of claim 80, said chimera polypeptide having a pI_{CHI} which differs from the pI_{API} so that at a precipitation pH, the polypeptide of interest can be separated from the chimera polypeptide by pH-modulated precipitation.

82. A method comprising expressing the chimera polypeptide of claim 80, cleaving the chimera polypeptide into a desired polypeptide and a pI modifier polypeptide, and separating the desired polypeptide from pI modifier polypeptide and from chimera polypeptide by pH-modulated precipitation.

83. A method comprising:
a. contacting the chimera polypeptide of claim 80 to an affinity binder which binds the chimera polypeptide to form an affinity binder-chimera polypeptide complex;
b. separating unwanted impurities from said affinity binder-chimera complex
c. cleaving chimera polypeptide to separate the desired polypeptide from the pI modifier polypeptide; and
d. isolating the desired polypeptide by pH-modulated precipitation.

84. A process comprising: expressing the DNA sequence of claim 49 to make a polypeptide comprising the polypeptide of SEQ. ID. NO. 2.


SEQUENCE LISTING (22 sequences)

SEQ ID NO: 1 - DNA, 96 bp, Artificial Sequence
  Synthetic nesiritide gene
agcccgaaaatggtgcagggcagcggctgcttcggccgcaaaatggatcgcatcagcagcagctcaggcctgggctgcaaagttctgcgccgtcat

SEQ ID NO: 2 - protein, 32 aa, Homo sapien
  DISULFID (10)..(26)
SPKMVZGSGCFGRKMDRISSSSGLGCKVLRRH

SEQ ID NO: 3 - DNA, 290 bp, Artificial Sequence
  synthetic nesiritide with fusion partner
atgggcggtgcgcatcatcatcatcatcacgtcgacaatgttgatgaaagctggctacaggaaggccagacgcgaattatttttgatggcgttaactcggcgtttcatctgtggattaatgggcgctgggtcggttacggccaggacagtcgtttgcccatgggcggtgcggtcgacgacgacgacaagagcccgaaaatggtgcagggcagcggctgcttcggccgcaaaatggatcgcatcagcagcagctcaggcctgggctgcaaagttctgcgccgtattaataa

SEQ ID NO: 4 - DNA, 28 bp, Artificial Sequence
  synthetic primer
tgcgtcttttactggctcttctcgctaa

SEQ ID NO: 5 - DNA, 36 bp, Artificial Sequence
  synthetic primer
agatctcgagctcggacattttcgggctcttgtcgt

SEQ ID NO: 6 - DNA, 33 bp, Artificial Sequence
  synthetic primer
ccagctcgagatgctgccctgcaccattttcgg

SEQ ID NO: 7 - DNA, 30 bp, Artificial Sequence
  synthetic primer
taccagctcgagagaagcagccgctgccct

SEQ ID NO: 8 - DNA, 36 bp, Artificial Sequence
  synthetic primer
catatggctcgagatccattttgcggccgaagcagc

SEQ ID NO: 9 - DNA, 34 bp, Artificial Sequence
  synthetic primer
atatggtaccagctgcgcagcccaggccgctgct

SEQ ID NO: 10 - DNA, 31 bp, Artificial Sequence
  synthetic primer
cactgcgtcttttactggctcttctcgctaa

SEQ ID NO: 11 - DNA, 37 bp, Artificial Sequence
  synthetic primer
gcttcgaattcccatatgcgcagaactttgcagccca

SEQ ID NO: 12 - DNA, 41 bp, Artificial Sequence
  synthetic primer
gccaaaacagccaagcttcttattaatgacggcgcagaact

SEQ ID NO: 13 - DNA, 39 bp, Artificial Sequence
  synthetic primer
gaactttgcagaggcctgagctgctgctgatgcgatcca

SEQ ID NO: 14 - DNA, 30 bp, Artificial Sequence
  synthetic primer
cgcatcagcagctcaggcctgggctgcaaa

SEQ ID NO: 15 - DNA, 24 bp, Artificial Sequence
  synthetic primer
tagctccttcggtcctccgatcgt

SEQ ID NO: 16 - DNA, 51 bp, Artificial Sequence
  synthetic primer
cccgttttttgggctaacaggaggaattaaccgtcgacaatgttgatgaaa

SEQ ID NO: 17 - DNA, 32 bp, Artificial Sequence
  synthetic primer
gatgatggtcgaccgcaccgcccatgggcaaa

SEQ ID NO: 18 - DNA, 33 bp, Artificial Sequence
  synthetic primer
agaaaccaattgtccatattgcatcagacattg

SEQ ID NO: 19 - DNA, 30 bp, Artificial Sequence
  synthetic primer
cgaccgcaccgacgtcgggcaaacgactgt

SEQ ID NO: 20 - DNA, 30 bp, Artificial Sequence
  synthetic primer
gacagtcgtttgcccgacgtcggtgcggtc

SEQ ID NO: 21 - DNA, 46 bp, Artificial Sequence
  synthetic primer
gcggctgcgttctgatttaatctcgagcaggctgaaaatcttctct

SEQ ID NO: 22 - DNA, 5982 bp, Unknown
  synthetic vector pET-RAZ-6-N
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcggtgcgcatcatcatcatcatcacgtcgacaatgttgatgaaagctggctacaggaaggccagacgcgaattatttttgatggcgttaactcggcgtttcatctgtggattaatgggcgctgggtcggttacggccaggacagtcgtttgcccgacgtcggtgcggtcgacgacgacgacgacaagagcccgaaaatggtgcagggcagcggctgcttcggccgcaaaatggatcgcatcagcagcagctcaggcctgggctgcaaagttctgcgccgtcattaataagaagcttggctgttttggcggatgagagaagattttcagcctgctcgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa